SEQUENCE LISTING (3 sequences)

SEQ ID NO: 1 - protein, 854 aa, Artificial Sequence
  synthetic polypeptide
  MISC_FEATURE 560: any non-polar amino acid Xaa
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVANKDKPLGAVALKSYEEELVKDPRIAATMENAQKGEIMPNIPQMSAFWYAVRTAVINAASGRQTVDEALKDAQTNSSSNNNNNNNNNNLGAATTGYDAVDDLLHYHERGNIQINGKDSFSNEQAGKFITRENQTWNGYKVFGQPVKLTFSFPDYKFSSTNVAGDTGLSKFSAEQQQQAKLSLQSWADVANITFTEVAAGQKANITFGNYSQDRPGHYDYGTQAYAFLPNTIWQGQDLGGQTWYNVNQSNVKHPATEDYGRQTFTHXIGHALGLSHPGDYNAGEGNPTYRDVTYAEDTRQFSLMSYWSETNTGGDNGGHYAAAPLLDDIAAIQHLYGANLSTRTGDTVYGFNSNTGRDFLSTTSNSQKVIFAAWDAGGNDTFDFSGYTANQRINLNEKSFSDVGGLKGNVSIAAGVTIENAIGGSGNDVIVGNAANNVLKGGAGNDVLFGGGGADELWGGAGKDIFVFSAASDSAPGASDWIRDFQKGIDKIDLSFFNKEAQSSDFIHFVDHFSGAAGEALLSYNASNNVTDLSVNIGGHQAPDFLVKIVGQVDVATDFIV

SEQ ID NO: 2 - protein, 854 aa, Artificial Sequence
  synthetic polypeptide
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVANKDKPLGAVALKSYEEELVKDPRIAATMENAQKGEIMPNIPQMSAFWYAVRTAVINAASGRQTVDEALKDAQTNSSSNNNNNNNNNNLGAATTGYDAVDDLLHYHERGNIQINGKDSFSNEQAGKFITRENQTWNGYKVFGQPVKLTFSFPDYKFSSTNVAGDTGLSKFSAEQQQQAKLSLQSWADVANITFTEVAAGQKANITFGNYSQDRPGHYDYGTQAYAFLPNTIWQGQDLGGQTWYNVNQSNVKHPATEDYGRQTFTHAIGHALGLSHPGDYNAGEGNPTYRDVTYAEDTRQFSLMSYWSETNTGGDNGGHYAAAPLLDDIAAIQHLYGANLSTRTGDTVYGFNSNTGRDFLSTTSNSQKVIFAAWDAGGNDTFDFSGYTANQRINLNEKSFSDVGGLKGNVSIAAGVTIENAIGGSGNDVIVGNAANNVLKGGAGNDVLFGGGGADELWGGAGKDIFVFSAASDSAPGASDWIRDFQKGIDKIDLSFFNKEAQSSDFIHFVDHFSGAAGEALLSYNASNNVTDLSVNIGGHQAPDFLVKIVGQVDVATDFIV

SEQ ID NO: 3 - DNA, 2580 bp, Artificial Sequence
  synthetic DNA
aagcttatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctgggtgcggcgaccaccggctacgacgcggttgacgacctgctgcactaccacgaacgcggcaatggcatccaaattaacggcaaagatagcttcagcaacgagcaggcgggtctgtttatcacccgtgaaaaccaaacctggaacggttacaaggtgtttggccagccggttaaactgaccttcagctttccggactataagttcagcagcaccaacgtggcgggtgataccggcctgagcaagtttagcgcggagcagcaacagcaagcgaaactgagcctgcagagctgggcggatgtggcgaacatcaccttcaccgaagttgcggcgggtcaaaaagcgaacattacctttggcaactacagccaggaccgtccgggtcactacgattatggcacccaagcgtatgcgttcctgccgaacaccatctggcagggtcaagacctgggtggccagacctggtacaacgtgaaccaaagcaacgttaagcacccggcgaccgaggattatggtcgtcagacctttacccacgcgattggtcatgcgctgggcctgagccatccgggtgactacaacgcgggcgagggcaacccgacctaccgtgacgtgacctatgcggaagatacccgtcagttcagcctgatgagctactggagcgaaaccaacaccggtggcgataacggtggccactatgcggcggcgccgctgctggatgatattgcggcgattcaacacctgtacggtgcgaacctgagcacccgtaccggtgacaccgtgtatggcttcaacagcaacaccggtcgtgattttctgagcaccaccagcaacagccagaaagttatctttgcggcgtgggatgcgggtggcaacgacaccttcgattttagcggttataccgcgaaccaacgtattaacctgaacgagaagagctttagcgatgttggtggcctgaagggtaacgtgagcatcgcggcgggcgttaccatcgaaaacgcgattggtggcagcggtaacgacgtgattgttggcaacgcggcgaacaacgtgctgaagggtggcgcgggtaacgacgttctgttcggtggcggtggcgcggatgagctgtggggtggcgcgggtaaagacatcttcgtgtttagcgcggcgagcgatagcgcgccgggtgcgagcgactggattcgtgatttccagaagggcatcgacaaaattgatctgagcttctttaacaaagaggcgcaaagcagcgacttcatccactttgttgatcactttagcggtgcggcgggtgaagcgctgctgagctacaacgcgagcaacaacgtgaccgacctgagcgttaacattggtggccaccaggcgccggattttctggtgaagattgtgggccaagtggatgttgcgaccgattttattgtgtaatgactcgag

(19) United States
(12) Patent Application Publication
(10) Pub. No.: US 2023/0044668 A1
(43) Pub. Date: Feb. 9, 2023

(54) UNIVERSAL ORAL DELIVERY DEVICE OF INTACT THERAPEUTIC POLYPEPTIDES WITH HIGH BIOAVAILABILITY

(71) Applicant: Panarum Corp, Dover, DE (US)

(72) Inventor: Milena Batalla, Buenos Aires (AR)

(73) Assignee: Panarum Corp, Dover, DE (US)

(21) Appl. No.: 17/433,576

(22) PCT Filed: Feb. 24, 2020
(86) PCT No.: PCT/US2020/019511

§ 371 Date: Aug. 24, 2021

Related U.S. Application Data
(60) Provisional application No. 62/819,670, filed on Mar. 17, 2019.
(60) Provisional application No. 62/809,687, filed on Feb. 24, 2019.

(51) Int. Cl. A61K 9/50; A61K 38/24; A61K 9/19

(52) U.S. Cl. CPC A61K 9/5078; A61K 38/24; A61K 9/19; C12Y 304/24

(57) ABSTRACT

The invention is related to the fully effective gastro-protected universal oral delivery device of gastro-protected nanoparticles for the transport of intact biologically active polypeptides into the circulatory system. This universal oral delivery device is made of gastro-protected nanoparticles that transport intact therapeutic polypeptides through the gastrointestinal system and it successfully performs the paracellular transepithelial passage of all therapeutic polypeptides from the intestinal lumen into the circulatory system, fully preserving the integrity and biological activity of those therapeutic polypeptides.

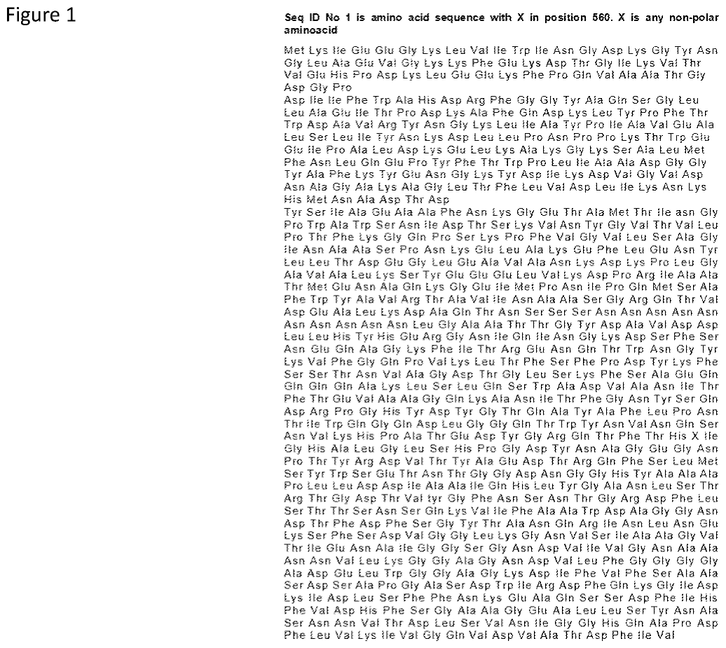

The Computer Readable Form (CRF) sequence listing ASCII text file filed via the USPTO's electronic filing system titled “REPLACEMENT 04-01-2022.txt” created on 04/04/2022 with a size of 17,895 bytes is incorporated by reference. The CRF is identical to the sequences shown in FIGS. 1-3.

### FIELD OF THE INVENTION

The present invention relates to the gastro-protected oral delivery device of intact therapeutic polypeptides into the circulatory system. The gastro-protected oral delivery device is formed by gastro-protected polypeptide nanoparticles. The gastro-protected polypeptide nanoparticles are formulated by a combination of therapeutic polypeptides, the purified recombinant polypeptide SERAR, a stabilizing polymer and a gastro-protected shell-coating with gastro-protective polymer. The purified recombinant polypeptide SERAR performs an innocuous temporary opening of the intestinal wall's intercellular junctions in vivo and performs the transepithelial paracellular passage of intact therapeutic polypeptides into the circulatory system. This oral delivery device successfully transports the intact therapeutic polypeptides contained in gastro-protected polypeptide nanoparticles through the gastrointestinal system and it successfully performs the releasing of the therapeutic polypeptides and purified recombinant polypeptide SERAR at controlled pH. SERAR recombinant polypeptide performs paracellular transepithelial passage of therapeutic polypeptide from the intestinal lumen into the circulatory system.

The present invention also relates to a method of producing the purified recombinant polypeptide SERAR, a method of producing gastro-protected polypeptide nanoparticles and a method of producing oral pharmaceutical composition containing gastro-protected polypeptide nanoparticles.

### REFERENCES

Smart A L et al., Oral peptide and protein delivery: intestinal obstacles and commercial prospects. Expert Opin Drug Deliv. 2014 August; 11(8):1323-35.
Lagassé H A et al., Recent advances in therapeutic protein drug development. F1000Research 2017, 6:113 7 Feb. 2017.
Philippart M et al., Oral Delivery of Therapeutic Proteins and Peptides: An Overview of Current Technologies and Recommendations for Bridging from Approved Intravenous or Subcutaneous Administration to Novel Oral Regimens, Oral Delivery of Therapeutic Proteins and Peptides Drug Res.
Abramson et al., An ingestible self-orienting system for oral delivery of macromolecules, Science 8 Feb. 2019: Vol. 363, Issue 6427, pp. 611-615.
Imran et al., U.S. Pat. No. 9,844,655 Dec. 19, 2017 Therapeutic agent preparations for delivery into a lumen of the intestinal tract using a swallowable drug delivery device
Mir Imran et al., U.S. Pat. No. 8,734,429 B2, May 27, 2014. Device, system and methods for the oral delivery of therapeutic compounds.
Kidron 2016 et al., U.S. Pat. No. 9,259,456 B2 Methods and composition for oral administration of proteins.
Casim Ali et al., PATENT WO 2012/122326 A1 SARKAR, Sep. 13, 2012 Engineered bacteria for oral delivery of glucoregulatory proteins
Gantier et al., U.S. Pat. No. 7,611,700 B2 (45), Nov. 3, 2009. Protease resistant modified interferon alpha polypeptides.
SHIMONI, Eyal et al., WO PATENT 2008/132727 A2, Nov. 6, 2008 Oral delivery of proteins and peptides.
Byun, Young-Ro et al., WO PATENT 02/087597A1 Oral delivery of macromolecules.
Michael et al., U.S. Pat. No. 6,613,332 B1 2003, Oral administration of therapeutic proteins.
Michael et al., U.S. Pat. No. 5,629,001 1997, Oral administration of therapeutic proteins.
Park, Ho-Yong et al., Patent EP 1 165 807 B1, May 13, 2009, A protease, a gene therefor and the use thereof.
M. Hiroshi, M. Kazuyuki, Serralysin and related bacterial proteinases, Methods and Enzymol. 248 (1995) 395-413.
Bhagat S, et al., Serratiopeptidase: a systematic review of the existing evidence.
Bhagat S, et. al. Int J Surg. 2013; 11(3):209-17.
Selan et al., Serratiopeptidase: a well-known metalloprotease with a new non-proteolytic activity against S. aureus biofilm, BMC Microbiology (2015) 15:207.
Chaudhari Snehal Anil et al., Production, characterization & optimization of potent protease (serratiopeptidase) from Serratia marcescens e 15. Int. Res J Pharm. App Sci., 2013; 3(4):95-98.
L. Jack Windsor and Darin L. Steele, Expression of Recombinant Matrix Metalloproteinases in Escherichia coli Chapter 4.
Tarlachkov S V et al., Cloning, purification and characterization of translationally fused protein DNA methyltransferase MbcircleHhal-EGFP, Process Biochemistry (2014).
Kaviyarasi N S et al., Characterization of a Gene Encoding Serrapeptidase from Serratia marcescens Strain (SRM) MTCC 8708, a Plant Isolate. Int. J. Curr. Microbiol. App. Sci (2015) 4(8): 206-214.
Nirale N M et al., Topical Formulations of Serratiopeptidase: Development and Pharmacodynamic Evaluation. Indian J Pharm Sci. 2010 January-February; 72(1): 65-71.
Kaviyarasi N S et al., Serrapeptidase gene of Serratia marcescens from plant origin expressed by Pichia pastoris has protease activity. Indian Journal of Medical Research and Pharmaceutical Sciences June 2016; 3(6).
Sylvie Letoffe et al., Cloning and Expression in Escherichia coli of the Serratia marcescens Metalloprotease Gene: Secretion of the Protease from E. coli in the Presence of the Erwinia chrysanthemi Protease Secretion Functions, Journal of Bacteriology, April 1991, p. 2160-2166.
Mohankumar A et al., Production and Characterization of Serratiopeptidase Enzyme from Serratia Marcescens. International Journal of Biology Vol. 3, No. 3; July 2011.
Nakahama K et al., Cloning and sequencing of Serratia protease gene, volume 14 Number 14 1986 Nucleic Acids Research.
Mike Tawse. The Miracle Enzyme® Is Serrapeptase, the 2nd Gift from Silkworms: Giving the answer to Pain, Inflammation, and Clear Arteries. 2002-2016 Naturally Healthy Publications.
Nitin Mali et al., Serratiopeptidase Loaded Chitosan Nanoparticles by Polyelectrolyte Complexation: In Vitro and In Vivo Evaluation. AAPS PharmSciTech, Vol. 16, No. 1, February 2015.
Shimada K et al., Extracellular Transport of Pseudoazurin of Alcaligenes faecalis in Escherichia coli Using the COOH-Terminal Domain of Serratia marcescens Serine Protease. J. Biochem. 116, 327-334 (1994).
Hamada K et al., Crystal Structure of Serratia Protease, a Zinc-Dependent Proteinase from Serratia sp. E-15, containing a fÁ-Sheet Coil Motif at 2.0 A Resolution. J. Biochem. 119, 844-851 (1996).
Qi et al., A single freeze-thawing cycle for highly efficient solubilization of inclusion body proteins and its refolding into bioactive form. Microbial Cell Factories 2015; 14: 24.
Vandana Gupte et al., Analytical techniques for serratiopeptidase: A review. Journal of Pharmaceutical Analysis, Volume 7, Issue 4, August 2017, Pages 203-207.
Selvarajan Ethiraj et al., Production, purification, characterization, immobilization, and application of Serrapeptase: a review. Front. Biol. 2017, 12(5): 333-348.
Tao et al., Cloning, Expression, and Purification of Insecticidal Protein Pr596 from Locust Pathogen Serratia marcescens HR-3. Curr Microbiol (2007) 55:228-233.
Moriya et al., Intestinal absorption of serrapeptase and its distribution to the inflammation sites. Article in Japanese Pharmacology and Therapeutics 31(8):659-666, January 2003.
Moriya N, et al., Intestinal absorption of serrapeptase (TSP) in rats. Biotechnol Appl Biochem 1994; 20:101-8.
Panagariya A et al., A preliminary trial of serratiopeptidase in patients with carpal tunnel syndrome. J Assoc Physicians India 1999; 47:1170-2.
Tachibana M, et al., A multi-centre, double-blind study of serrapeptase versus placebo in post-antrotomy buccal swelling. Pharmatherapeutica 1984; 3:526-30.
Michael et al., U.S. Pat. No. 5,629,001 1997. Oral administration of therapeutic proteins for treatment of infectious disease.
Desai et al., U.S. Pat. No. 5,916,596A 1999. Protein stabilized pharmacologically active agents, methods for the preparation thereof and methods for the use thereof.
Dunn et al., U.S. Pat. No. 5,990,194A 1999. Biodegradable in-situ forming implants and methods of producing the same.
Michael and Litwin, U.S. Pat. No. 6,613,332B1 2003. Oral administration of therapeutic proteins.
Samaritani and Donati, U.S. Pat. No. 7,740,884B2 2010. Freeze-dried FSH/LH formulations.
Kidron M., U.S. Pat. No. 9,259,456B2 2016. Methods and composition of oral administration of proteins.
Taneja and Singh, Rational design of polysorbate 80 stabilized human serum albumin nanoparticles tailored for high drug loading and entrapment of Irinotecan, Int J Pharm. 536 (2017) 82-94.
Jahanban-Esfahlan et al., A simple improved desolvation method for the rapid preparation of albumin nanoparticles, Int J Biol Macromol. 91 (2016) 703-709.
Lagassé et al., Recent advances in (therapeutic protein) drug development, F1000Res. 6 (2017) 1-17.
Smart et al., Oral peptide and protein delivery: intestinal obstacles and commercial prospects, Expert Opin Drug Deliv. 11 (2014) 1323-1335.
Shakeri et al., Preparation of protein-loaded PLGA-PVP blend nanoparticles by nanoprecipitation method: entrapment, Initial burst and drug release kinetic studies, Nanomedicine Journal. 2 (2015) 175-186.
Dadparvar et al., Freeze-drying of H1-6-loaded recombinant human serum albumin nanoparticles for improved storage stability, Eur J Pharm Biopharm. 88 (2014) 510-517.
Varshosaz et al., Eudragit nanoparticles loaded with silybin: a detailed study of preparation, freeze-drying condition and in vitro/in vivo evaluation, J Microencapsul. 32 (2014) 211-223.
Ochi et al., Development of nanocrystal formulation of meloxicam with improved dissolution and pharmacokinetic behaviors, Int J Pharm. 474 (2014) 151-156.
Passot et al., Effect of Product Temperature During Primary Drying on the Long-Term Stability of Lyophilized Proteins, Pharm Dev Technol. 12 (2014) 543-553.
Galisteo-González and Molina-Bolivar, Systematic study on the preparation of BSA nanoparticles. Colloids Surf. B: Biointerfaces, Colloids Surf B Biointerfaces. 123 (2014) 286-292.
Ahuja et al., Diclofenac-loaded Eudragit S100 nanosuspension for ophthalmic delivery J Microencapsul. 28 (2011) 37-45.
Karsdal et al., Lessons Learned From the Development of Oral Calcitonin: The First Tablet Formulation of a Protein in Phase III Clinical Trials, J Clin Pharmacol. 51 (2011) 460-471.
MacLachlan et al., A Scalable, Extrusion-Free Method for Efficient Liposomal Encapsulation of Plasmid DNA, Pharmaceutical Research. 22 (2005) 460-471.
He et al., The transport mechanisms of polymer nanoparticles in Caco-2 epithelial cells, Biomaterials. 34 (2013) 6082-6098.
Cetin et al., Formulation and In vitro Characterization of Eudragit® L100 and Eudragit® L100-PLGA Nanoparticles Containing Diclofenac Sodium, AAPS PharmSciTech. 11 (2010) 1250-1256.
Takada et al., Influence of Ethanol on Physical State of Freeze-Dried Mannitol, Pharm Res. 26 (2009) 1112-1120.
Anhorn et al., Freeze drying of human serum albumin (HSA) nanoparticles with different excipients, Int J Pharm. 363 (2008) 162-169.
Sandhya et. al., Liposomal Formulations of Serratiopeptidase: In Vitro Studies Using PAMPA and Caco-2 Models, Molecular Pharmaceutics. 5 (2008) 92-97.
Damgé et. al., Nanoparticle strategies for the oral delivery of insulin, Christiane, Expert Opin. Drug Deliv. 5 (2008) 45-68.
Han et al., Effects of Sugar Additives on Protein Stability of Recombinant Human Serum Albumin during Lyophilization and Storage, Archives of Pharmacol Research. 30 (2007) 1124-1131.
Abdelwahed et al., Freeze-drying of nanoparticles: Formulation, process and storage considerations, Adv Drug Deliv Rev. 58 (2006) 1688-1713.
Tang and Pikal, Design of Freeze-Drying Processes for Pharmaceuticals: Practical Advice, Pharm Res. 21 (2004) 191-200.
Langer et al., Optimization of the preparation process for human serum albumin (HSA) nanoparticles, Int J Pharm. 257 (2003) 169-180.
Wang, Lyophilization and development of solid protein pharmaceuticals, Int J Pharm. 203 (2000) 1-60.
Okada et al., Drug delivery using biodegradable microspheres, Journal of Controlled Release. 28 (1994) 121-129.
Pikal, Formulation and Delivery of Proteins and Peptides; Chapter 8: Freeze-Drying of Proteins, Process, Formulation, and Stability. (1994) 120-133.
Mangroliya Rohit Kumar et. al., Formulation design and in vitro characterization of tablet a combination of etodolac and serratiopeptidase. World Journal of Pharmacy and Pharmaceutical Sciences. Volume 6, Issue 1, 672-687.
A Prameela Rani et. al. Enteric dispersion of Serratiopeptidase with Eudragit L100 and Formulation of Controlled Release Tablets of Serratiopeptidase. International Journal of PharmTech. Research Vol. 12, No. 02, pp 139-144, 2019.
PanKaj nerkar et. al. Development and in vitro evaluation of Eudragit RLPO based polymeric nanoparticles of Lansoprazole. Indian Journal of Novel drug delivery 8 (3), July-September, 2016, 165-174.
Nicholas G. Lamson, Anionic nanoparticles enable the oral delivery of proteins by enhancing intestinal permeability, Nature Biomedical Engineering (2019) 4 Nov. 2019.
Sam Maher et. al. Application of Permeation Enhancers in Oral Delivery of Macromolecules: An Update. Pharmaceutics 2019, 11, 41.
Sung Hee Lee et. al. Intestinal Permeability Regulation by Intercellular Junction: Implication on Inflammatory Bowel Diseases. Intestinal Research 2015; 13(1):11-18.
Aaron Buckley et. al. Cell Biology of Intercellular Junction Barrier Regulation and Mucosal Disease. Cold Spring Harb Perspect Biol.; 10(1), 2018 Sep. 27.
Mary Carmen Valenzano et. al. Remodeling of Intercellular Junctions and Enhancement of Barrier Integrity of the CACO-2 Intestinal Epithelial Cell Layer by Micronutrients. PLOS ONE, Jul. 30, 2015.
Qinghua Yu et. al. The effect of various absorption enhancers on tight junction in the human intestinal Caco-2 cell line. Drug Development and Industrial Pharmacy, 2013; 39(4): 587-592.

### SUMMARY OF THE INVENTION

The present invention relates to a gastro-protected oral delivery device of therapeutic polypeptides into the bloodstream of a patient, the device comprises of therapeutic polypeptides and the SERAR purified recombinant polypeptide.

In different embodiments, proportions and composition may vary.

This gastro-protected oral delivery device successfully transports intact therapeutic polypeptides contained in gastro-protected polypeptide nanoparticles through the gastrointestinal system. The gastro-protected polypeptide nanoparticles fully release the therapeutic polypeptides and the purified recombinant protein SERAR at intestinal pH 5-6 (here pH is the scale measuring how acid or basic a water-based solution is). Purified recombinant protein SERAR triggers an innocuous temporary opening of the intestinal wall's intercellular junctions and accomplishes the fully paracellular transepithelial passage of therapeutic polypeptide from the intestinal lumen into the circulatory system. The fully paracellular transepithelial passage of therapeutic proteins from the intestinal lumen into the circulatory system preserve their structure and biological activity ensure the high bioavailability.

Transepithelial paracellular passage of therapeutic polypeptide from the lumen of the intestinal epithelium into the circulatory system is achieved by the presence of the purified recombinant polypeptide SERAR alongside the therapeutic polypeptide. This purified recombinant polypeptide SERAR actively triggers an innocuous temporary opening of the intestinal epithelium's intercellular junctions. This purified recombinant polypeptide SERAR comprises an amino acids sequence derived from the sequence of serratiopeptidase with its proteolytic activity suppressed.

Therapeutic polypeptide/s and the recombinant purified polypeptide SERAR are co-formulated as gastro-protected polypeptide nanoparticles in a water dispersion by solvent injection with selected gastro-protective polymers (examples include but are not limited to methacrylic acid-methacrylate copolymers).

The combination of therapeutic polypeptides and this recombinant purified polypeptide SERAR with a gastro-protective polymer is generated by desolvation.

The obtained polypeptide nanoparticles are gastro-protected with a shell coating with selected gastro-protective polymers (examples include but are not limited to methacrylic acid-methacrylate copolymers or copolymers of acrylic and methacrylic acid esters containing quaternary ammonium groups).

This invention also includes the synthesis of pharmaceutical composition of liquid and solid oral formulations of the gastro-protected polypeptide nanoparticles in therapeutic amounts.

An embodiment of the present invention provides the sequence of the purified recombinant purified polypeptide SERAR that is co-formulated alongside the therapeutic polypeptide in the polypeptide nanoparticles. In a preferred embodiment SERAR recombinant polypeptide sequence derived from sequence SEQ ID No 1 whose shows a non-polar amino acid in position 560. In another exemplary embodiment, the SERAR sequence is SEQ ID No 2, comprising alanine in position 560.

In embodiments this application indicates that the therapeutic polypeptides that are part of the invention are comprised in the group of recombinant polypeptides, naturally-existing biologically active proteins, fusion proteins, hormones, growth factors, plasma proteins, coagulation factors, polypeptide vaccines, toxins and other protein antigens, monoclonal antibodies, and replacement enzymes and peptides.

In preferred embodiments of the present invention the therapeutic polypeptides are comprised monoclonal antibodies and fusion polypeptides including but not limited to rituximab, adalimumab, infliximab, trastuzumab, ranibizumab, pertuzumab, denosumab, cetuximab, bevacizumab, nivolumab, pembrolizumab, eculizumab, ustekinumab, golimumab, omalizumab, pembrolizumab and combinations of two or more of these.

In a preferred embodiment of the present invention the therapeutic polypeptides are recombinant proteins including but not limited to follicle stimulating hormone, luteinizing hormone, chorionic gonadotropin hormone, erythropoietin, GCS-F, filgrastim, somatotropin, betalFN1a, betalFN1b, alphalFN2a, alphalFN2b, interleukin 2, etanercept, insulin, eptacog alfa, human recombinant Factor VII, human recombinant Factor VIII, human recombinant Factor XII, human recombinant Factor XIII, alfa-agalsidase, alglucerase, beta-agalsidase, imiglucerase, taliglucerase-alfa, velaglucerase-alfa, laronidase, idulsurfase, elosulfase-alfa, galsulfase, alglucosidase-alfa and combinations of two or more of these.

In some embodiment this application provides a nucleic acid comprising a sequence that encodes a polypeptide comprising the amino acid sequence of SEQ ID NO. 2, or of a fragment of SEQ ID NO. 2 of 18 bases or larger.

In another embodiment of the present invention is an expression vector comprising the nucleic acid coding for SEQ ID No 3, or any fragment of it that is 18 consecutive bases long or larger, operably linked to an expression control sequence, a cultured cell comprising such vector, a cultured cell comprising the nucleic acid coding for SEQ ID NO. 3, or any fragment of it that is 18 consecutive bases long or larger, operably linked to an expression control sequence. And it also provides a cultured cell transfected with such vector, or a progeny of said cell, wherein the cell expresses the polypeptide coded by SEQ ID NO. 1 or any fragment of it that is 18 consecutive bases long or larger.

In some embodiment this application provides a method of producing the purified recombinant polypeptide SERAR, comprising the culture of the above-mentioned cell under conditions that successfully achieves the expression of the recombinant polypeptide SERAR and the obtaining of the purified recombinant polypeptide SERAR from the cell or the medium of the cell.

In some embodiment this application provides an oral delivery device of intact therapeutic polypeptides into circulatory system comprising a purified recombinant polypeptide SERAR and therapeutic polypeptide/s, in which the polypeptides are co-formulated as gastro-protected nanoparticles.

In some embodiment according to the delivery device, this application provides a method for the synthesis of protein nanoparticles comprising the injection of an ethanolic solution of gastro-protective polymer into an aqueous solution containing the purified recombinant polypeptide SERAR one or more therapeutic polypeptides and a stabilizing agent. The size of such nanoparticles can range between 50 and 1000 nanometers, and is more commonly in the range between 250 and 350 nanometers.

In some embodiment relating to the nanoparticles production method a narrow tube is used for the injection of the ethanolic solution into the aqueous solution containing the purified recombinant polypeptide SERAR and one or more therapeutic polypeptides. In some embodiments the ethanolic solution is injected at a flow range comprised between 0.5 and 5000 mL/min, and in some embodiments the flow range is comprised between 2 and 160 mL/min. In some embodiments the nanoparticles suspension is further diluted with the addition of a stabilizing agent, and more commonly such stabilizing agent is polyvinylpyrrolidone. In some embodiment the dilution factor by addition of the stabilizing agent is comprised between 0.5 and 30, and more commonly this factor is 1 or higher.

In some embodiment of the polypeptide nanoparticles synthesis methods there is provided the use of a gastro-protective polymer comprised in the group of the anionic copolymers, being the composition of the anionic copolymer comprised in the group of methacrylic acid and methyl methacrylate.

In some embodiment such nanoparticles solution is diafiltrated and concentrated using tangential flow filtration.

In some embodiment of the nanoparticles synthesis method water miscible organic solvent is removed from the mixture, wherein the removal of the solvent may be done by dialysis, ultra-filtration, solvent evaporation at a reduced pressure, N2 current evaporation or tangential flow filtration.

In some embodiment regarding the nanoparticles production method it is mentioned that nanoparticles suspension is freeze dried. In some embodiment of the polypeptide nanoparticles freeze-dryed with or without lyoprotectant agent in the polypeptide nanoparticles suspension before freeze drying, examples of such lyoprotectant agents is comprised but no limited to the group of sucrose, lactose and mannitol.

In some embodiment it is provided a pharmaceutical composition containing gastro-protected therapeutic polypeptide nanoparticles of oral delivery to a human subject comprising the synthesis of freeze dried gastro-protected polypeptide nanoparticles and their further formulation as oral solid and/or liquid dosage forms such as powder, paper, granules, rigid capsules, flexible capsules, pearls, tablets, film-coated tablets, extracts, solutions, potions, emulsions and suspensions.

### BRIEF DESCRIPTION OF THE DRAWINGS

### Figures

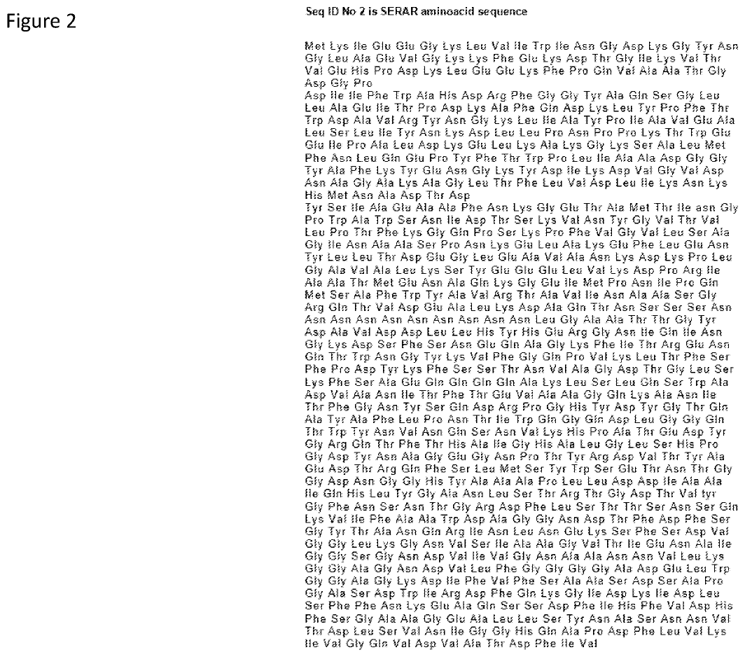

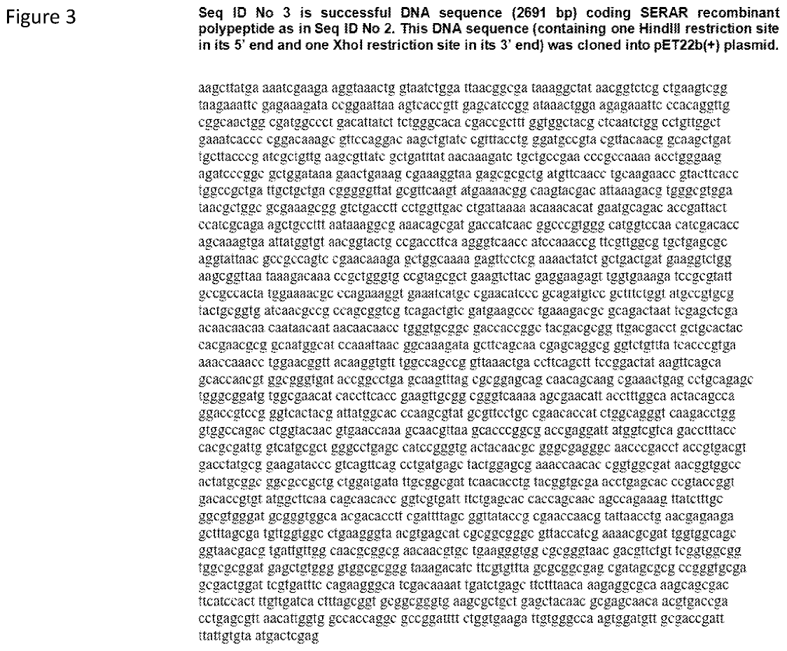

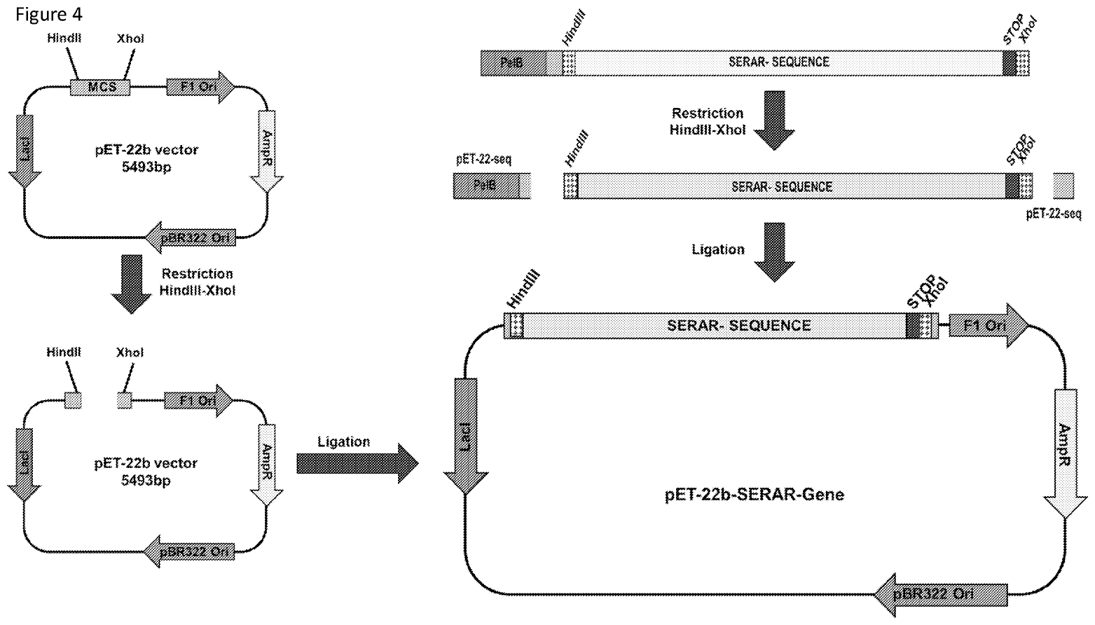

FIG. 1:
Seq ID No 1 is amino acid sequence with X in position 560. X is any non-polar aminoacid
Met Lys Ile Glu Glu Gly Lys Leu Val Ile Trp Ile Asn Gly Asp Lys Gly Tyr Asn
Gly Leu Ala Glu Val Gly Lys Lys Phe Glu Lys Asp Thr Gly Ile Lys Val Thr Val
Glu His Pro Asp Lys Leu Glu Glu Lys Phe Pro Gln Val Ala Ala Thr Gly Asp
Gly Pro
Asp Ile Ile Phe Trp Ala His Asp Arg Phe Gly Gly Tyr Ala Gln Ser Gly Leu Leu
Ala Glu Ile Thr Pro Asp Lys Ala Phe Gln Asp Lys Leu Tyr Pro Phe Thr Trp
Asp Ala Val Arg Tyr Asn Gly Lys Leu Ile Ala Tyr Pro Ile Ala Val Glu Ala Leu
Ser Leu Ile Tyr Asn Lys Asp Leu Leu Pro Asn Pro Pro Lys Thr Trp Glu Glu Ile
Pro Ala Leu Asp Lys Glu Leu Lys Ala Lys Gly Lys Ser Ala Leu Met Phe Asn
Leu Gln Glu Pro Tyr Phe Thr Trp Pro Leu Ile Ala Ala Asp Gly Gly Tyr Ala Phe
Lys Tyr Glu Asn Gly Lys Tyr Asp Ile Lys Asp Val Gly Val Asp Asn Ala Gly Ala
Lys Ala Gly Leu Thr Phe Leu Val Asp Leu Ile Lys Asn Lys His Met Asn Ala
Asp Thr Asp
Tyr Ser Ile Ala Glu Ala Ala Phe Asn Lys Gly Glu Thr Ala Met Thr Ile asn Gly
Pro Trp Ala Trp Ser Asn Ile Asp Thr Ser Lys Val Asn Tyr Gly Val Thr Val Leu
Pro Thr Phe Lys Gly Gln Pro Ser Lys Pro Phe Val Gly Val Leu Ser Ala Gly Ile
Asn Ala Ala Ser Pro Asn Lys Glu Leu Ala Lys Glu Phe Leu Glu Asn Tyr Leu
Leu Thr Asp Glu Gly Leu Glu Ala Val Ala Asn Lys Asp Lys Pro Leu Gly Ala Val
Ala Leu Lys Ser Tyr Glu Glu Glu Leu Val Lys Asp Pro Arg Ile Ala Ala Thr Met
Glu Asn Ala Gln Lys Gly Glu Ile Met Pro Asn Ile Pro Gln Met Ser Ala Phe Trp
Tyr Ala Val Arg Thr Ala Val Ile Asn Ala Ala Ser Gly Arg Gln Thr Val Asp Glu
Ala Leu Lys Asp Ala Gln Thr Asn Ser Ser Ser Asn Asn Asn Asn Asn Asn Asn
Asn Asn Asn Leu Gly Ala Ala Thr Thr Gly Tyr Asp Ala Val Asp Asp Leu Leu
His Tyr His Glu Arg Gly Asn Ile Gln Ile Asn Gly Lys Asp Ser Phe Ser Asn Glu
Gln Ala Gly Lys Phe Ile Thr Arg Glu Asn Gln Thr Trp Asn Gly Tyr Lys Val Phe
Gly Gln Pro Val Lys Leu Thr Phe Ser Phe Pro Asp Tyr Lys Phe Ser Ser Thr
Asn Val Ala Gly Asp Thr Gly Leu Ser Lys Phe Ser Ala Glu Gln Gln Gln Gln Ala
Lys Leu Ser Leu Gln Ser Trp Ala Asp Val Ala Asn Ile Thr Phe Thr Glu Val Ala
Ala Gly Gln Lys Ala Asn Ile Thr Phe Gly Asn Tyr Ser Gln Asp Arg Pro Gly His
Tyr Asp Tyr Gly Thr Gln Ala Tyr Ala Phe Leu Pro Asn Thr Ile Trp Gln Gly Gln
Asp Leu Gly Gly Gln Thr Trp Tyr Asn Val Asn Gln Ser Asn Val Lys His Pro Ala
Thr Glu Asp Tyr Gly Arg Gln Thr Phe Thr His X Ile Gly His Ala Leu Gly Leu
Ser His Pro Gly Asp Tyr Asn Ala Gly Glu Gly Asn Pro Thr Tyr Arg Asp Val Thr
Tyr Ala Glu Asp Thr Arg Gln Phe Ser Leu Met Ser Tyr Trp Ser Glu Thr Asn Thr
Gly Gly Asp Asn Gly Gly His Tyr Ala Ala Ala Pro Leu Leu Asp Asp Ile Ala Ala
Ile Gln His Leu Tyr Gly Ala Asn Leu Ser Thr Arg Thr Gly Asp Thr Val tyr Gly
Phe Asn Ser Asn Thr Gly Arg Asp Phe Leu Ser Thr Thr Ser Asn Ser Gln Lys
Val Ile Phe Ala Ala Trp Asp Ala Gly Gly Asn Asp Thr Phe Asp Phe Ser Gly Tyr
Thr Ala Asn Gln Arg Ile Asn Leu Asn Glu Lys Ser Phe Ser Asp Val Gly Gly Leu
Lys Gly Asn Val Ser Ile Ala Ala Gly Val Thr Ile Glu Asn Ala Ile Gly Gly Ser Gly
Asn Asp Val Ile Val Gly Asn Ala Ala Asn Asn Val Leu Lys Gly Gly Ala Gly Asn
Asp Val Leu Phe Gly Gly Gly Gly Ala Asp Glu Leu Trp Gly Gly Ala Gly Lys Asp
Ile Phe Val Phe Ser Ala Ala Ser Asp Ser Ala Pro Gly Ala Ser Asp Trp Ile Arg
Asp Phe Gln Lys Gly Ile Asp Lys Ile Asp Leu Ser Phe Phe Asn Lys Glu Ala Gln
Ser Ser Asp Phe Ile His Phe Val Asp His Phe Ser Gly Ala Ala Gly Glu Ala Leu
Leu Ser Tyr Asn Ala Ser Asn Asn Val Thr Asp Leu Ser Val Asn Ile Gly Gly His
Gln Ala Pro Asp Phe Leu Val Lys Ile Val Gly Gln Val Asp Val Ala Thr Asp Phe
Ile Val
FIG. 2:
Seq ID No 2 is SERAR aminoacid sequence
Met Lys Ile Glu Glu Gly Lys Leu Val Ile Trp Ile Asn Gly Asp Lys Gly Tyr Asn
Gly Leu Ala Glu Val Gly Lys Lys Phe Glu Lys Asp Thr Gly Ile Lys Val Thr Val
Glu His Pro Asp Lys Leu Glu Glu Lys Phe Pro Gln Val Ala Ala Thr Gly Asp
Gly Pro
Asp Ile Ile Phe Trp Ala His Asp Arg Phe Gly Gly Tyr Ala Gln Ser Gly Leu Leu
Ala Glu Ile Thr Pro Asp Lys Ala Phe Gln Asp Lys Leu Tyr Pro Phe Thr Trp
Asp Ala Val Arg Tyr Asn Gly Lys Leu Ile Ala Tyr Pro Ile Ala Val Glu Ala Leu
Ser Leu Ile Tyr Asn Lys Asp Leu Leu Pro Asn Pro Pro Lys Thr Trp Glu Glu Ile
Pro Ala Leu Asp Lys Glu Leu Lys Ala Lys Gly Lys Ser Ala Leu Met Phe Asn
Leu Gln Glu Pro Tyr Phe Thr Trp Pro Leu Ile Ala Ala Asp Gly Gly Tyr Ala Phe
Lys Tyr Glu Asn Gly Lys Tyr Asp Ile Lys Asp Val Gly Val Asp Asn Ala Gly Ala
Lys Ala Gly Leu Thr Phe Leu Val Asp Leu Ile Lys Asn Lys His Met Asn Ala
Asp Thr Asp
Tyr Ser Ile Ala Glu Ala Ala Phe Asn Lys Gly Glu Thr Ala Met Thr Ile asn Gly
Pro Trp Ala Trp Ser Asn Ile Asp Thr Ser Lys Val Asn Tyr Gly Val Thr Val Leu
Pro Thr Phe Lys Gly Gln Pro Ser Lys Pro Phe Val Gly Val Leu Ser Ala Gly Ile
Asn Ala Ala Ser Pro Asn Lys Glu Leu Ala Lys Glu Phe Leu Glu Asn Tyr Leu
Leu Thr Asp Glu Gly Leu Glu Ala Val Ala Asn Lys Asp Lys Pro Leu Gly Ala
Val Ala Leu Lys Ser Tyr Glu Glu Glu Leu Val Lys Asp Pro Arg Ile Ala Ala Thr
Met Glu Asn Ala Gln Lys Gly Glu Ile Met Pro Asn Ile Pro Gln Met Ser Ala Phe
Trp Tyr Ala Val Arg Thr Ala Val Ile Asn Ala Ala Ser Gly Arg Gln Thr Val Asp
Glu Ala Leu Lys Asp Ala Gln Thr Asn Ser Ser Ser Asn Asn Asn Asn Asn Asn
Asn Asn Asn Asn Leu Gly Ala Ala Thr Thr Gly Tyr Asp Ala Val Asp Asp Leu
Leu His Tyr His Glu Arg Gly Asn Ile Gln Ile Asn Gly Lys Asp Ser Phe Ser Asn
Glu Gln Ala Gly Lys Phe Ile Thr Arg Glu Asn Gln Thr Trp Asn Gly Tyr Lys Val
Phe Gly Gln Pro Val Lys Leu Thr Phe Ser Phe Pro Asp Tyr Lys Phe Ser Ser
Thr Asn Val Ala Gly Asp Thr Gly Leu Ser Lys Phe Ser Ala Glu Gln Gln Gln
Gln Ala Lys Leu Ser Leu Gln Ser Trp Ala Asp Val Ala Asn Ile Thr Phe Thr
Glu Val Ala Ala Gly Gln Lys Ala Asn Ile Thr Phe Gly Asn Tyr Ser Gln Asp Arg
Pro Gly His Tyr Asp Tyr Gly Thr Gln Ala Tyr Ala Phe Leu Pro Asn Thr Ile Trp
Gln Gly Gln Asp Leu Gly Gly Gln Thr Trp Tyr Asn Val Asn Gln Ser Asn Val
Lys His Pro Ala Thr Glu Asp Tyr Gly Arg Gln Thr Phe Thr His Ala Ile Gly His
Ala Leu Gly Leu Ser His Pro Gly Asp Tyr Asn Ala Gly Glu Gly Asn Pro Thr
Tyr Arg Asp Val Thr Tyr Ala Glu Asp Thr Arg Gln Phe Ser Leu Met Ser Tyr
Trp Ser Glu Thr Asn Thr Gly Gly Asp Asn Gly Gly His Tyr Ala Ala Ala Pro
Leu Leu Asp Asp Ile Ala Ala Ile Gln His Leu Tyr Gly Ala Asn Leu Ser Thr Arg
Thr Gly Asp Thr Val tyr Gly Phe Asn Ser Asn Thr Gly Arg Asp Phe Leu Ser
Thr Thr Ser Asn Ser Gln Lys Val Ile Phe Ala Ala Trp Asp Ala Gly Gly Asn
Asp Thr Phe Asp Phe Ser Gly Tyr Thr Ala Asn Gln Arg Ile Asn Leu Asn Glu
Lys Ser Phe Ser Asp Val Gly Gly Leu Lys Gly Asn Val Ser Ile Ala Ala Gly Val
Thr Ile Glu Asn Ala Ile Gly Gly Ser Gly Asn Asp Val Ile Val Gly Asn Ala Ala
Asn Asn Val Leu Lys Gly Gly Ala Gly Asn Asp Val Leu Phe Gly Gly Gly Gly
Ala Asp Glu Leu Trp Gly Gly Ala Gly Lys Asp Ile Phe Val Phe Ser Ala Ala Ser
Asp Ser Ala Pro Gly Ala Ser Asp Trp Ile Arg Asp Phe Gln Lys Gly Ile Asp Lys
Ile Asp Leu Ser Phe Phe Asn Lys Glu Ala Gln Ser Ser Asp Phe Ile His Phe
Val Asp His Phe Ser Gly Ala Ala Gly Glu Ala Leu Leu Ser Tyr Asn Ala Ser
Asn Asn Val Thr Asp Leu Ser Val Asn Ile Gly Gly His Gln Ala Pro Asp Phe
Leu Val Lys Ile Val Gly Gln Val Asp Val Ala Thr Asp Phe Ile Val
FIG. 3
Seq ID No 3 is successful DNA sequence (2691 bp) coding SERAR recombinant polypeptide
as in Seq ID No 2. This DNA sequence (containing one HindiII restriction site in its 5’ end and
one XhoI restriction site in its 3’ end) was cloned into pET22b(+) plasmid.
aagcttatga aaatcgaaga aggtaaactg gtaatctgga ttaacggcga taaaggctat aacggtctcg ctgaagtcgg
taagaaattc gagaaagata ccggaattaa agtcaccgtt gagcatccgg ataaactgga agagaaattc ccacaggttg
cggcaactgg cgatggccct gacattatct tctgggcaca cgaccgcttt ggtggctacg ctcaatctgg cctgttggct
gaaatcaccc cggacaaagc gttccaggac aagctgtatc cgtttacctg ggatgccgta cgttacaacg gcaagctgat
tgcttacccg atcgctgttg aagcgttatc gctgatttat aacaaagatc tgctgccgaa cccgccaaaa acctgggaag
agatcccggc gctggataaa gaactgaaag cgaaaggtaa gagcgcgctg atgttcaacc tgcaagaacc gtacttcacc
tggccgctga ttgctgctga cgggggttat gcgttcaagt atgaaaacgg caagtacgac attaaagacg tgggcgtgga
taacgctggc gcgaaagcgg gtctgacctt cctggttgac ctgattaaaa acaaacacat gaatgcagac accgattact
ccatcgcaga agctgccttt aataaaggcg aaacagcgat gaccatcaac ggcccgtggg catggtccaa catcgacacc
agcaaagtga attatggtgt aacggtactg ccgaccttca agggtcaacc atccaaaccg ttcgttggcg tgctgagcgc
aggtattaac gccgccagtc cgaacaaaga gctggcaaaa gagttcctcg aaaactatct gctgactgat gaaggtctgg
aagcggttaa taaagacaaa ccgctgggtg ccgtagcgct gaagtcttac gaggaagagt tggtgaaaga tccgcgtatt
gccgccacta tggaaaacgc ccagaaaggt gaaatcatgc cgaacatccc gcagatgtcc gctttctggt atgccgtgcg
tactgcggtg atcaacgccg ccagcggtcg tcagactgtc gatgaagccc tgaaagacgc gcagactaat tcgagctcga
acaacaacaa caataacaat aacaacaacc tgggtgcggc gaccaccggc tacgacgcgg ttgacgacct gctgcactac
cacgaacgcg gcaatggcat ccaaattaac ggcaaagata gcttcagcaa cgagcaggcg ggtctgttta tcacccgtga
aaaccaaacc tggaacggtt acaaggtgtt tggccagccg gttaaactga ccttcagctt tccggactat aagttcagca
gcaccaacgt ggcgggtgat accggcctga gcaagtttag cgcggagcag caacagcaag cgaaactgag cctgcagagc
tgggcggatg tggcgaacat caccttcacc gaagttgcgg cgggtcaaaa agcgaacatt acctttggca actacagcca
ggaccgtccg ggtcactacg attatggcac ccaagcgtat gcgttcctgc cgaacaccat ctggcagggt caagacctgg
gtggccagac ctggtacaac gtgaaccaaa gcaacgttaa gcacccggcg accgaggatt atggtcgtca gacctttacc
cacgcgattg gtcatgcgct gggcctgagc catccgggtg actacaacgc gggcgagggc aacccgacct accgtgacgt
gacctatgcg gaagataccc gtcagttcag cctgatgagc tactggagcg aaaccaacac cggtggcgat aacggtggcc
actatgcggc ggcgccgctg ctggatgata ttgcggcgat tcaacacctg tacggtgcga acctgagcac ccgtaccggt
gacaccgtgt atggcttcaa cagcaacacc ggtcgtgatt ttctgagcac caccagcaac agccagaaag ttatctttgc
ggcgtgggat gcgggtggca acgacacctt cgattttagc ggttataccg cgaaccaacg tattaacctg aacgagaaga
gctttagcga tgttggtggc ctgaagggta acgtgagcat cgcggcgggc gttaccatcg aaaacgcgat tggtggcagc
ggtaacgacg tgattgttgg caacgcggcg aacaacgtgc tgaagggtgg cgcgggtaac gacgttctgt tcggtggcgg
tggcgcggat gagctgtggg gtggcgcggg taaagacatc ttcgtgttta gcgcggcgag cgatagcgcg ccgggtgcga
gcgactggat tcgtgatttc cagaagggca tcgacaaaat tgatctgagc ttctttaaca aagaggcgca aagcagcgac
ttcatccact ttgttgatca ctttagcggt gcggcgggtg aagcgctgct gagctacaac gcgagcaaca acgtgaccga
cctgagcgtt aacattggtg gccaccaggc gccggatttt ctggtgaaga ttgtgggcca agtggatgtt gcgaccgatt
ttattgtgta atgactcgag

### DETAILED DESCRIPTION

This invention relates to a gastro-protected oral delivery device comprised of therapeutic polypeptides and a purified recombinant polypeptide SERAR and defined later on, the device deliver the therapeutic polypeptides by transepithelial paracellular passage with high bioavailability.

The gastro-protected oral delivery device successfully transports therapeutic polypeptides in polypeptide nanoparticles through the gastrointestinal system. Furthermore, said device successfully performs the paracellular transepithelial passage of intact therapeutic polpypeptide/s from the intestinal lumen into the circulatory system.

The transepithelial paracellular passage of therapeutic polypeptides through the intestinal epithelium into the circulatory system is achieved by the presence of the SERAR purified recombinant polypeptide alongside the therapeutic polypeptide.

The sequence of the SERAR recombinant polypeptide is derived from Seq ID No 1 (FIG. 1). SERAR recombinant polypeptide is specific amino acid sequence described in Seq ID No 2 (FIG. 2) and it comprises a specific amino acid sequence that actively triggers a temporary opening of the intestinal epithelium's intercellular junctions, a linker and a sequence from a maltose-binding protein domain.

The SERAR purified recombinant polypeptide triggers a temporary opening of the intestinal epithelium's intercellular junctions is derived from the sequence of serratiopeptidase (Spep), an extracellular metalloprotease produced by Serratia marcescens ATCC 21074 (E-15), and has been modified in order to eliminate its proteolytic activity. The serratiopeptidase enzyme (Serratia marcescens E15 protease) is an oral anti-inflammatory supplement with EC number 3.4.24.40 having a molecular weight of approximately 52 kDa (kilodaltons). Serratiopeptidase was first isolated from enterobacterium Serratia sp, a microorganism originally isolated in the late 1960s from silk worm Bombyx mori. Serratiopeptidase is present in the silk worm intestine and allows the emerging moth to dissolve its cocoon. It is produced by purification from fermentation of Serratia marcescens or Serratia sp. E 15. The Serratiopeptidase enzyme belongs to the Serralysin group of enzymes, it is a proteolytic enzyme with many favorable biological properties like anti-inflammatory, analgesic, anti-bacterial, fibrinolytic properties. Moreover, serratiopeptidase with enteric coating is widely used by oral administration in clinical practice for the treatment of many diseases.

The purified recombinant polypeptide SERAR actively triggers an innocuous temporary opening of the intestinal epithelium's intercellular junctions in vitro and in vivo. Furthermore, it is fully effective in the transepithelial paracellular passage activity of therapeutic polypeptides in vitro and in vivo, without the proteolytic activity of the wild type serratiopeptidase, and the maltose binding protein (MBP) that grants correct folding of the SERAR polypeptide.

The crux of the present invention is the gastro-protected oral delivery device of the SERAR recombinant polypeptide and at least one therapeutic polypeptide. The SERAR recombinant polypeptide comprises the specific amino acid sequence of SEQ ID NO. 2 that performs innocuous temporary opening of the intestinal epithelium's intercellular junctions and therapeutic polypeptides pass through the intestinal epithelium by paracellular transepithelial passage in vitro and in vivo.

In a first embodiment of the present invention a universal oral delivery device, the device delivering intact therapeutic polypeptides into the circulatory system in a subject. The universal oral device is comprised of a purified recombinant polypeptide, including but not limiting to the SERAR purified recombinant polypeptide (SEQ ID NO. 2), and at least one or a combination of more therapeutic polypeptides. In other embodiments of the present invention the universal oral device is gastro-protected and also comprises a group consisting of at least two gastro-protective polymers.

Embodiments of the present invention may comprise different therapeutic polypeptides, descriptions including but not limited to:
at least one therapeutic polypeptide in the group of therapeutic polypeptides are comprised in the group of: recombinant polypeptides, naturally-existing biologically active proteins, fusion proteins, hormones, growth factors, plasma proteins, coagulation factors, toxins and other protein antigens, monoclonal antibodies, replacement enzymes and peptides.
at least one therapeutic polypeptide in the group of therapeutic polypeptides is a monoclonal antibody comprised in the group rituximab, adalimumab, infliximab, trastuzumab, ranibizumab, pertuzumab, denosumab, cetuximab, bevacizumab, nivolumab, pembrolizumab, eculizumab, ustekinumab, golimumab, omalizumab, pembrolizumab.
at least one therapeutic polypeptide is a recombinant protein comprised in the group of: follicle stimulating hormone, luteinizing hormone, Chorionic gonadotrophic hormone, erythropoietin, filgrastim, molgramostim, somatotropin, betalFN1a, betalFN1b, IFNalpha2a, IFNalpha2b, interleukin 2, etanercept, ranibizumab, insulin, eptacog alfa, human recombinant Factor VII, human recombinant Factor VIII, human recombinant Factor XII, human recombinant Factor XIII, alfa-agalsidase, alglucerase, beta-agalsidase, imiglucerase, taliglucerase-alfa, velaglucerase-alfa, laronidase, idulsurfase, elosulfase-alfa, galsulfase, alglucosidase-alfa and combinations of two or more of these.

Embodiment of this invention includes a nucleic acid sequence that encodes a SERAR recombinant polypeptide comprising the amino acid sequence of SEQ ID NO. 2 or of a fragment of SEQ ID NO. 2 of 18 bases or larger. Another embodiment of the present invention comprises an expression vector with the nucleic acid sequence of SERAR recombinant polypeptide (Seq. ID No 3). Another embodiment of the present invention includes a cultured cell, wherein the expression vector of SERAR acid nucleic sequence is described above. The nucleic acid sequence Seq ID No. 3 is operably linked to an expression control sequence, where this sequence express the SERAR recombinant polypeptide Seq ID. No 2.

In other embodiments of the present invention, the gastro-protected oral delivery device is comprised of gastro-protected polypeptide nanoparticles formulated by a combination of therapeutic polypeptides, purified recombinant polypeptide SERAR Seq ID. No 2, a gastro-protective polymer, and a stabilizing polymer.

Other embodiments of the present invention include methods for preparing polypeptides including but not limited to Seq. ID no 1. In one embodiment, a method for producing recombinant polypeptide SERAR (SEQ ID NO: 2) comprises the steps of cultivating bacteria carrying plasmids with genes coding for the desired SERAR recombinant polypeptide.

A method for constructing plasmids containing pre-specified genes and controlling DNA sequences, including but not limited to the DNA sequence of the SERAR polypeptide, is given by the so-called recombinant DNA technique.

It is known in the art that it is possible to obtain, from the cultivated bacteria cells carrying such recombinant plasmid DNA, gene-coded proteins which inherently are characteristic to other organisms than the bacterium used as host cell. In the preparation of recombinant plasmid DNA, a so-called cloning vector (that is, a plasmid which is able to replicate in the host bacterium) is combined with a DNA fragment containing a gene, many genes, DNA sequences or expression cassettes coding for the pre-specified gene polypeptide/s, and/or controlling its expression. The recombinant DNA technique, in its most useful form, is based on the following principle:

Assume we pre-selected a plasmid vector that is a circular DNA molecule that contains a multiple cloning site with several sites for restriction endonucleases cleavage, other DNA expression control units and at least one selection marker (a DNA fragment coding for resistance to an antibiotic, for example). The vector is treated with one or more restriction enzymes to produce one or several species of a linear molecule.

On the other hand, we pre-selected a different DNA sample which has been treated with the same endonuclease.

DNA is cut into pieces in a very specific way by restriction endonucleases including but not limited to HindIII and XhoI. These pieces are joined to each other by DNA ligase in order to obtain molecules composed of the vector to which a foreign DNA fragment has been fused, and which are called recombinant DNA.

An alternative or complementary method involves one extra step before ligation by DNA ligase, which is the completion of the cohesive ends generated by the restriction endonuclease by using a DNA Polymerase and nucleotides. This performs for ligation of fragments that do not have matching cohesive ends.

Next, we insert the resulting plasmid into a pre-selected bacterial host cell using either electroporation or calcium chloride transformation. Bacteria that incorporated the plasmid are selected by using the selection marker that was included in the plasmid (for example, resistance to a certain antibiotic, for example, ampicillin, kanamycin). Bacteria that incorporated the plasmid are resistant to the antibiotic and grow in a medium that contains such antibiotic. The larger the number of plasmids per bacterial cell, the highest the resistance to the antibiotic and the higher the chances of such cell of producing the protein of interest in high amounts. Expression of the desired protein coded in the plasmid is granted since the selection marker is included in the same plasmid, adjacent to the DNA fragment that codes for the protein of interest.

Another embodiment of the present invention includes a method for producing recombinant polypeptide SERAR (SEQ ID NO: 2) comprising a step of cultivating bacteria carrying plasmids with genes coding for the desired SERAR recombinant polypeptide, being such genes cloned in a plasmid sensitive to changes in temperature, in which the plasmid allows both effective plasmid amplification and production of large quantities of the plasmid gene product. The method uses plasmid with a temperature-dependent plasmid copy number pattern and shows a controlled constant plasmid copy number when host bacteria are cultivated at a certain temperature. On the other hand, when the host bacteria carrying the plasmid is grown at a different temperature, a plasmid copy number pattern occurs a higher number of plasmids per cell.

Hence, the number of copies of such plasmid is low at one temperature, which is an advantage since it decreases the risk that the cloned plasmid or its gene products could disturb the growing of the host bacterium. However, the amount of the plasmid is rapidly increased by a simple temperature shift, whereby simultaneous formation of the cloned plasmid and its gene products are obtained, and the production of plasmid's gene products proceeds rapidly.

The cultured cell per se is successfully performed using the productivity conventional techniques, including conventional chemically defined nutritive media which, with the productivity for the bacterial species in question, and also, the harvesting of the gene products is performed in accordance with productivity methods to the identity of the gene products of the present invention. An Special and critical parameter of the production process of the gene product is the temperature regulation including at least a period of cultivation to achieve a temperature at which the plasmid shows the plasmid shows an increasing in the copy number. This pattern includes a period of cultivation in which the plasmid is copied in a high number of copies, and gene product of the plasmid are formed in higher amounts.

The culture of the transformed E. coli cells is grown at 37° C., and ampicillin is added to a concentration (q.s. 100 μg/ml) which it inhibits the growth of cells containing plasmids with normal copy number. Isopropyl β-D-1-thiogalactopyranoside (IPTG) is added to the cells in order to induce the expression of the gene and the production of the SERAR recombinant polypeptide Seq. ID No 2. The culture of the plasmid-containing cells is then grown at 34° C.

Cells cultured as mentioned above are disrupted and centrifugated to obtain the SERAR inclusion bodies (IBs). These SERAR IBs are washed and resolubilized in lysis buffer and solubilization buffer with 6 M UREA, respectively. Cell pellets are then frozen at −20° C. and then centrifuged at 12000 rpm. The protein of interest is effectively refolded with 20 mM TRIS-HCl sucrose 8% pH 8.0.

Embodiments of the present invention include an expression vector, which is a plasmid. Said plasmid comprises at least one restriction endonuclease: only one site susceptible to cleavage by the endonuclease. Moreover, said site is such that after insertion of a fragment of foreign DNA at this site, the resulting recombinant DNA replicates autonomously by the temperature-regulated signal of the present invention, increasing the production of the number of identical copies of plasmids regulated by temperature.

The most suitable restriction endonucleases used in recombinant DNA technology are those giving the so-called “cohesive ends” on both the cloning vector and the DNA fragment, in other words, single-stranded regions at the ends of the molecules with complementary base sequence allowing base pairing to identical sequences. The cloning vector constructed contains a DNA sequence Seq ID No. 3 coding for SERAR recombinant polypeptide Seq. ID No. 2 comprising: a) maltose binding protein sequence and b) the specific amino acid sequence of serratiopeptidase with the modified amino acid in position 560.

The cloning vector contains genes mediating a so-called marker, useful for identification and/or selection of cells carrying the plasmid. The most useful marker is an antibiotic resistance-related gene, for example, ampicillin resistance, since this is used for an easy counter selection of bacteria that have not received the recombinant plasmid after treatment for transforming a recombinant DNA into a bacterial host.

Embodiments of the present invention of gastro-protected universal oral delivery device of therapeutic polypeptides into the circulatory system of a human subject. This gastro-protected universal oral delivery device formulated as gastro-protected polypeptide nanoparticles overcome the gastrointestinal tract barriers and are an effective oral delivery device of therapeutic polypeptides into circulatory system using different kinds of oral pharmaceutical compositions (e.g. liquid, solid or a combination of both).

The purified recombinant polypeptide called SERAR Seq. ID No 2 comprising: a) an artificially mutated amino acid sequence from Serratiopeptidase (Serratia E15 protease) that is involved in the effective transepithelial paracellular passage activity by opening the intestinal epithelium's intercellular junction in vitro and in vivo without proteolytic activity due to the substitution of a glutamic acid in the catalytic site with another amino acid, including but not limited to, a residue of alanine; and b) a stabilizing domain from maltose binding protein (MBP).

An embodiment of the present invention includes a method for obtaining protein nanoparticles comprising the therapeutic polypeptide and the SERAR polypeptide. Polypeptide nanoparticles are synthesized by nanoprecipitation using a gastro-protective polymer dissolved in an organic solvent injected in an aqueous solution composed by a) at least one therapeutic polypeptide and; b) the SERAR purified recombinant polypeptide Seq ID No 2.

The protein nanoparticle synthesis method starts from buffer solutions containing the SERAR polypeptide, at least one therapeutic polypeptide, a gastro-protective polymer and a stabilizing hydrophilic polymer. The stabilizing hydrophilic polymer is, but is not limited to, polyvinylpyrrolidone. The resulting solution, composed of an appropriate ratio of SERAR polypeptide between 10 to 30 mg and one therapeutic dose of the therapeutic polypeptide, is stabilized at room temperature.

A water-miscible organic solvent solution which is, but not limited to, ethanol, is injected from above into the protein buffer solution at a rate of 2 to 160 milliliters per minute (mL/min) under constant stirring. The ethanolic solution is composed by a gastro-protective polymer or copolymer (for example, acrylic acid with methacrylate). The gastro-protective polymer, dissolved in an ethanol solution, is injected to the protein buffer solution producing a decrease in the solubility of the proteins and thus, the formation of protein nanoparticles by nanoprecipitation with efficiency over 99%. Later a solution of a hydrophilic polymer (e.g. polyvinylpyrrolidone) is added in order to stabilize the protein nanoparticles.

Finally, the ethanolic solvent is eliminated by different methods, including but not limited to, dialysis, ultra-filtration, evaporation at reduced pressure, evaporation with N2 gas injection, or tangential flow filtration.

According to the present invention, the protein nanoparticles size can range from 50 to 1000 nm, with method variations that can narrow the distribution to an average size population between 250 and 350 nm, are dispersed in an aqueous solution and they act as a vehicle carrying the therapeutic polypeptides (naturally occurring or recombinant proteins, active ingredients of biological origin, including fusion proteins, protein hormones, growth factors, protein vaccines, plasma proteins, coagulation factors, toxins and other protein antigens, monoclonal antibodies, bites and replacement enzymes).

The therapeutic polypeptides used may be already commercialized and they can be obtained from the purification of human fluids or biotechnological production.

The synthesized protein nanoparticles may be used directly as liquid dispersion. Alternatively, they may be conserved through a freeze-drying process in the presence or absence of lyoprotectant agents (e.g. sugars, amino acids, polyols, glycerol, or peptides), and then re-dispersed in a liquid medium preserving the proteins nanoparticles shape and size.

An embodiment of the present invention involves a method for the creation of a the gastro-protected polypeptide nanoparticles wherein these are generated by a gastro-protective shell coating of the polypeptide nanoparticles with a gastro-protective polymer that preserves intact the therapeutic polypeptides from the stomach-intestinal conditions. The correct gastro-protection ratio of polypeptide nanoparticles is a shell coating from 1:3 to 1:9 weight/weight (w/w) gastro-protective polymer.

An embodiment of the present invention involves a method for the creation of the oral administration to a subject, the gastro-protected oral delivery device of therapeutic polypeptides successfully formulated in gastro-protected polypeptide nanoparticles are formulated in oral pharmaceutical composition. The oral pharmaceutical composition used are liquid oral dosage forms (e.g. solutions, syrups or elixirs) or solid oral dosage forms (e.g. granules, tablets, filled hard or softgel capsules, or extemporal solids).

An embodiment of the present invention involves a method for the generation of a syrup pharmaceutical composition. According to this method, the protein nanoparticle dispersion (containing the encapsulated therapeutic polypeptide/s) is homogenized within the sucrose syrup containing, or not, preservative agents. Next, the preparation is conditioned in the proper container and is dosed by volume. The syrup formulation is appropriate when the dose of therapeutic protein is bigger than 500 mg/dose and ease of swallowing is an issue for pediatric, elder, hospitalized patients.

Another embodiment of the present invention involves a method for the manufacture of an oral solid pharmaceutical composition. Firstly, this oral solid pharmaceutical composition is based on the gastro-protected oral delivery device of therapeutic polypeptides Layering. This layering consisting of the first coating with the gastro-protected oral delivery device of therapeutic polypeptides over sugar micro/spheres to obtain the vehicle system. Secondly, an Enteric Coating with a gastro-protective polymer to preserve the vehicle system from the digestion of said composition in a stomach-intestine of a subject, and thirdly, the double-coated microspheres are dosage and loaded within gelatin capsules.

The coating processes are performed on the sugar microspheres with an average size between 300 and 1000 micrometers (μm) and/or standard spheres from 0.5 to 2 millimeters. The coating processes consist of a fluid bed coater with a bottom-up spraying system (Würster) and constant aerodynamic convection at up to 40° C. for drying and protection of the polypeptides involved in the process. The Pharm-a-spheres® (Evonik Industries AG) brand represents an example of sucrose micro/spheres that may be used for this step. This manufacturing method is appropriate for those therapeutic proteins administered at very low doses (in the order of micrograms, or μg in symbols) and the fluid bed coating process control the loading of protein nanoparticles over the sugar microspheres. This method uses a fluid bed coater which is loaded with the sucrose micro/spheres and these are thermostabilized at up to 40° C. The coating suspension is then pumped (using, for example, a peristaltic pump).

The first coating suspension (the gastro-protected oral delivery device of therapeutic polypeptides layering suspension) is composed by a) the gastro-protected polypeptide nanoparticles dispersion containing the therapeutic polypeptide/s, b) an adhesion polymer (for example 4-7% w/v polyvinylpyrrolydone or hydroxypropyl cellulose) (here w/v refers to weight over volume in grams and milliliters, e.g., 1% w/v 1 gram of sugar in 100 mL of water), c) talc or colloidal silica (c.a. 3% w/v) for the prevention of the electrostatic and auto-adhesion of micro/spheres and d) distilled water as solvent. The gastro-protected polypeptide nanoparticles layered micro/spheres are dried for 10 min. Later, the gastro-protected polypeptide nanoparticles coated micro/spheres are subjected with a second gastro-protective coating with a gastro-protective polymer.

The choice of the polymer (30-40% w/w of total solids) depends on the kind of controlled delivery or the site of releasing where the therapeutic protein is aimed to be absorbed. For example, the Eudragit® polymers commercialized by Evonik Industries AG, such as L 100-5S or L 30 D-55 are used for delivery at duodenum, the L 100 or L 12.5 for delivery at jejunum, the S 100, S 12.5 or FS 30 D are used for delivery at ileum and colon.

The second gastro-protected coating suspension also contains a) a plasticizer (1-5% w/v, for example triethyl citrate, trioctyl citrate, triehexyl citrate and acetylated monoglycerides) for diminishing the glass transition temperature of the gastro-protective polymer, b) talc (6-7% w/v), c) colored or transparent lacquer and c) distilled water as solvent. The micro/spheres are dried again for 10 min. Both coating suspensions (gastro-protected polypeptide nanoparticles layering and gastro-protective coating) have c.a. 5-15% w/v solid concentration giving a favorable viscosity for the spraying application.

The final microspheres with the double coating, gastro-protected polypeptide nanoparticles layered and the gastro-protective coating are dosed and filled in capsules for oral administration to a subject. The dose of the therapeutic polypeptide determines the microsphere size, the therapeutic polypeptide concentration in the gastro-protected polypeptide nanoparticles layering suspension and the capsule size.

Next, experiments are reproduced where different embodiments of the present invention take place. The first three examples include the SERAR recombinant polypeptide obtention. The following examples show the use of the SERAR as the universal oral delivery. The gastro-protected polypeptides nanoparticles dispersion obtained is administered directly as an oral liquid pharmaceutical composition and/or lyophilized for liquid or solid oral pharmaceutical compositions (e.g. extemporal suspensions, syrups, liquid-filled hard capsules, soft capsules, coated microspheres filled capsules, and granulates).

### EXAMPLES

The following examples are included for explanatory purposes only and no limit to the result of the invention. The examples demonstrate generic processes.

### Example 1

### Expression Vector Obtention and Clone Selection of SERAR

Target DNA sequence (SEQ ID No 3, introduced in FIG. 3) coding for SERAR recombinant polypeptide (SEQ ID No 2) is successfully synthesized. The synthesized sequence is cloned into vector pET-22b(+), containing a ampicillin-resistance gene, by using two restriction endonucleases (HindIII and XhoI) with unique sites within the plasmid for protein expression in E. coli.

For evaluation of the expression the E. coli strain BL21(DE3) is transformed with the recombinant plasmid and cultured in plates with ampicillin. A single colony from such plate is inoculated into chemically defined medium containing ampicillin; culture is incubated at 37° C. at 200 rpm and then induced 4 hours with IPTG. SDS-PAGE and capillary gel electrophoresis is used to monitor the expression amount of the SERAR recombinant polypeptide successfully obtained.

### Example 2

### Obtention of SERAR Purified Recombinant Polypeptide

Production of the purified recombinant polypeptide SERAR (SEQ ID NO: 2) comprises the cultivation of bacteria carrying plasmids with genes coding for the desired recombinant protein. The method for producing the recombinant polypeptide SERAR comprises growth of bacteria carrying a plasmid for recombinant expression of such polypeptide, induced with IPTG which must be added to the culture medium. The bacteria master cell bank (MCB) is manufactured by amplification of the bacteria containing the expression plasmid, and vials of such bank are used as seed for preparation of the inoculum.

Presence of expression plasmid in the bacterium is confirmed by isolation and identification via sequencing.

### Inoculum:

10 Erlenmeyer flasks containing 100 mL of LB medium with ampicillin 100 μg/mL are inoculated with 90 μl from one vial of the MCB each. All flasks are incubated at 34° C. for 15.5 h with continuous orbital shaking at 250 rpm.

After this incubation, samples from each flask are taken and OD600 nm is measured to each sample to determine bacterial concentration. Microscopic observation is also performed to discard contamination.

OD values above 3.0 are obtained and the content of all Erlenmeyer's are pooled to a final volume of 1000 mL and OD=3.41.

### Fed-Batch Cultivation in Bioreactor:

A bioreactor with a working volume of 15 L is used, and 10 L of chemically defined medium added to the vase. The following parameters are set: 400-800 rpm agitation, higher than 30% pO₂, 1-2 vvm aeration, pH 7.00 and 34° C. temperature.

Initial values after inoculation are: OD 0.38, 1 vvm, 400 rpm, pH 7.03 and pO₂ 84%.

Agitation and aeration are adjusted according to the O₂ consumption during the culture in order to maintain the desired parameters.

After 3 h of culture the OD is 4.52 and glucose concentration is 1 g/L. Induction is started in this moment by addition of yeast extract and 50 mL of IPTG 1 M.

Glucose 40% feed is initiated at 3.5 h from the start to maintain pO₂ above 30% in a cascade mode, with a set up concentration of glucose of 1 g/L.

The total induction period is 4 h, and samples are collected every hour. Maximum OD 14.08 is reached at 3 h post induction. Final OD after 7.5 h of process is 13.86, with a maximum velocity of growth during the exponential phase of the culture. Total wet biomass or wet pellet obtained from the 10 L of working volume is 180 g. Total glucose added during the fed-batch is 112 g.

### Harvest and Clarification:

Total culture volume (10 L) is centrifuged at 17500 g and 4° C. for 15 min. The pellet obtained (180 g) contains mostly intact bacteria, and is stored at −70° C. until further use.

### Bacterial Rupture and Separation of Inclusion Bodies:

The total amount of wet pellet obtained in harvest (180 g) is resuspended in 2 L of Buffer Tris 20 mM, NaCl 200 mM, EDTA 1 mM, pH 7.4 until total dissolution. OD of such solution is 66.7.

The total volume of resuspended pellet is subjected to 4 serial homogenization cycles. Product obtained is centrifuged at 9500 g at 4° C. for 45 min, and supernatant is separated for purification of SERAR in soluble fraction (2 L).

Pellets containing inclusion bodies (72 g) are washed with Buffer Tris 20 mM, NaCl 200 mM, EDTA 1 mM, pH 7.4, and centrifuged at 9500 g at 4° C. for 45 min. Two further washing steps with Tris 20 mM Urea 4M are executed, and one final wash step with Buffer Tris 20 mM, NaCl 200 mM, EDTA 1 mM, pH 7.4 is performed. Supernatant is discarded and pellets (12 g) are stored at −70° C. for further processing.

Total distribution of the recombinant polypeptide SERAR is 80% in IBs and 20% in the soluble fraction determined by densitometric SDS-PAGE and capillary gel electrophoresis.

Higher than 98% of the protein content of the pellets is recovered as SERAR recombinant polypeptide.

### Solubilization of Inclusion Bodies (IB) by Denaturation:

IBs containing 8.5 g of total protein are fully solubilized with 6 M urea at a concentration of 20 g of pellet per liter of buffer, followed by magnetic stirring at 900 rpm for 30 min.

This solubilized IBs is stored at −20° C.

Refolding of SERAR from Inclusion Bodies:

The dissolved IBs in 6 M urea solution from the previous step are centrifuged at 4000 g at 4-8° C. for 20 min. The recovered supernatant contains SERAR recombinant polypeptide and the pellet is discarded.

The supernatant is dropped at a constant rate of 50 mL/min into a vessel containing refolding buffer TrisHCl 20 mM sucrose 8% pH 7.4 with mild magnetic stirring.

The supernatant is finally diluted 10 times with the refolding buffer, and pH is adjusted to 6.0 with acetic acid.

This mixture is maintained for 48 h at 4-8° C. with mild magnetic stirring.

After 48 h the refolding volume is diafiltered against 10 volumes of Tris HCl 20 mM pH 7.4 using TFF with a molecular cut off of 10 kDa.

Concentration is also adjusted to 4-6 g/L and purity obtained is higher than 90%.

SERAR Recombinant Polypeptide Purified and Refolded from Inclusion Bodies:

SERAR recombinant polypeptide solution from the refolding step is subjected to chromatography with Capto DEAE resin.

Protein solution containing the SERAR polypeptide already folded at a concentration of 4.3 g/L in a Tris 20 mM pH7.4 buffer is loaded in the column at a concentration of 30 mg of protein per mL of resin.

Resin has been previously sanitized with NaOH 0.1 N and equilibrated in Tris 20 mM pH 8.0

Sample is loaded at a 150 cm/h linear flow followed by 3 column volumes (CVs) of wash buffer composed of Tris 20 mM NaCl 50 mM pH 8.0.

Elution is performed at the same linear flow, with Elution buffer (Tris 20 mM NaCl 1 M pH 8.0) ranging from 0 to 100% of Elution buffer in 10 CVs.

The recombinant polypeptide SERAR starts eluting with 0.4 M NaCl up to 0.55 M NaCl.

Purity of SERAR recombinant polypeptide in such eluate is 98% and the yield of the process is higher than 89%.

### Example 3

Transepithelial Paracellular Passage of FSH with SERAR Study:

### Cell Culture:

Caco-2 cell line HTB-37 (ATCC, Rockville, Md.), derived from human colon cells, and is used for all experiments. Cells are maintained in Dulbecco's Modified Eagles Medium (DMEM, American Type Culture Collection (ATCC), Rockville, Md.) supplemented with 67 IU/mL of penicillin, 67 μg/L of streptomycin, and 100 mL/L of fetal bovine serum. Monolayers are grown on Bio FIL-24 well pore 1 μm translucent PET membrane filter supports according to supplier instructions. At the end of the growth period, the integrity of the cell monolayer is confirmed by transepithelial electrical resistance (TEER) measurements (Millicell-ERS Voltohm meter, Millipore, Billerica, Mass.).

TEER Measures the Opening of Intercellular Junctions with Different Concentrations of SERAR:

Upper filter supports containing viable Caco-2 monolayers are transferred into a 24-well cell culture plate and 1000 μL of media is dispensed into each basolateral compartment. Solutions containing the recombinant FSH 11.000 mUI/ml (Gonal F®) and different doses of SERAR polypeptide solution (0.42, 1, 3, 6, 9, 12, 15 mg) are applied to the apical compartment and TEER readings are taken at each time 0 min, 20 min, 40 min, 1 h, 2 h, 3 h, 4 h, 5 h and 6 h.

### Transepithelial Paracellular Passage Study:

Firstly, purified SERAR recombinant polypeptide and FSH are dissolved in Dulbecco's Modified Eagles Medium (DMEM, American Type Culture Collection (ATCC), Rockville, Md.).

rFSH and different concentrations of SERAR Solutions are added to the apical side of Caco-2 monolayers. Samples are taken from the basolateral compartment at time: 0 min, 20 min, 40 min, 1 h, 2 h, 3 h, 4 h, 5 h and 6 h and Transepithelial Paracellular Passage is quantified by the amount of rFSH transported across the barrier in the basolateral well through the time by IMMULITE. Positive control experiments are performed with addition of rFSH+EDTA 2.5 mM on the apical section of the cells.

### Results

### SERAR Recombinant Polypeptide Opens the Intercellular Junctions and Reduces the TEER of the Caco-2 Monolayer:

Using TEER as a surrogate marker for FSH permeability, the study of all doses of the SERAR purified recombinant polypeptide is studied. The use of TEER as a measurement for permeability has several advantages, including convenience and a lack of dependence on the size of the solute, thereby ensuring the generality of results.

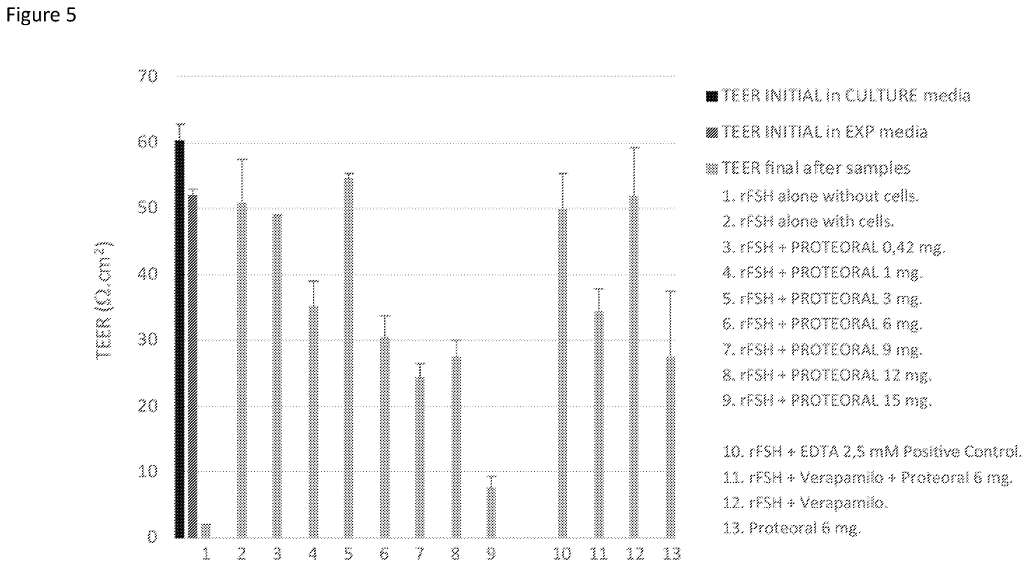

SERAR recombinant polypeptide opens the intercellular junctions and reduces the TEER of the Caco-2 monolayer after 20 min of exposure to SERAR with a reversible mechanism. SERAR exhibited maximal reduces the TEER of the Caco-2 monolayer at the dose of 15 mg in FIG. 5.

### SERAR Performs the Transepithelial Paracellular Passage of FSH:

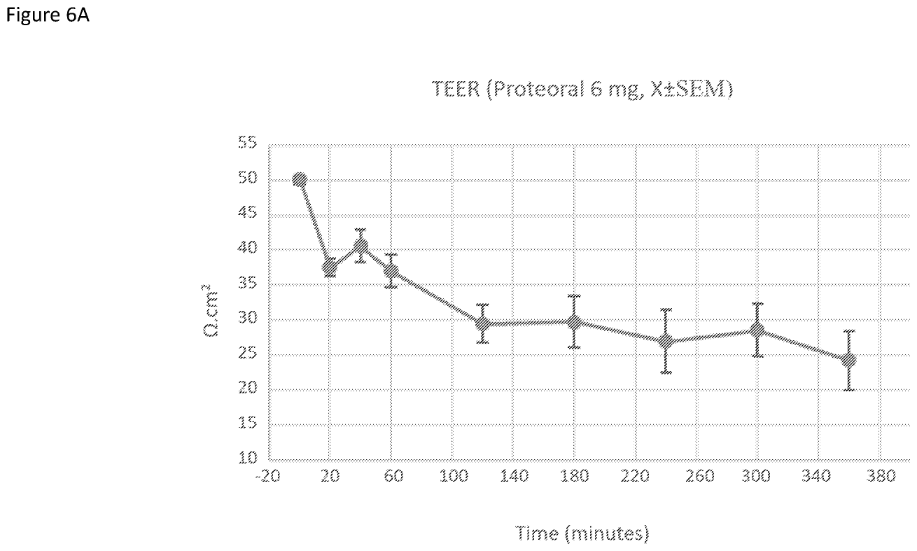

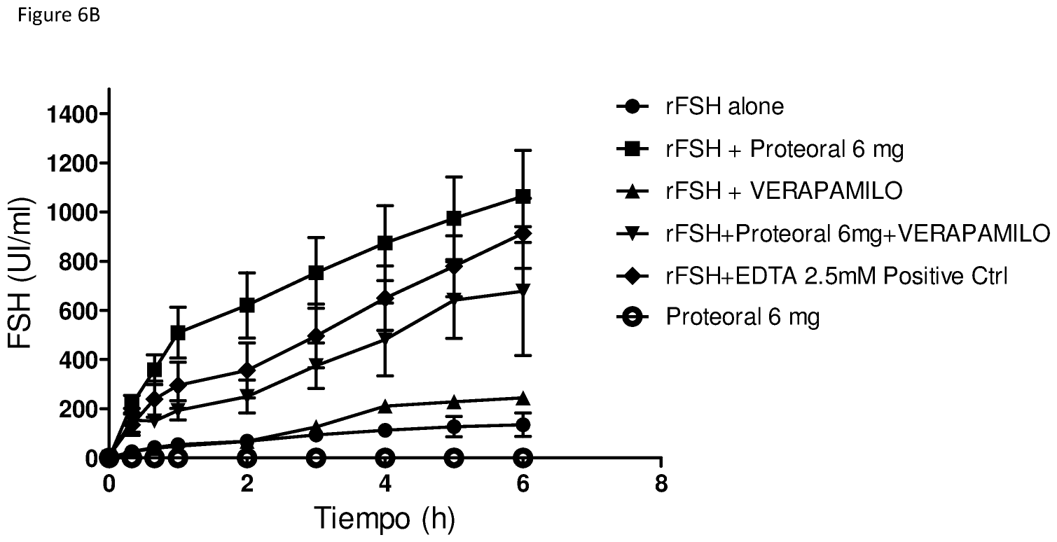

SERAR exhibited pronounced reduces the TEER of the Caco-2 monolayer at the dose of 6 mg at different times during the study in FIG. 6A.

The purified recombinant polypeptide SERAR is effective transepithelial paracellular passage device validated by methods used in this example. The device increases the transepithelial paracellular passage of the therapeutic polypeptides molecules, such us the glycoprotein Follicle Stimulating Hormone (FSH) of 30 kDa, more than 1000 times. These values are better than the maximum attainable permeability achieved, indeed better than a positive control EDTA 2.5 mM. This serves as an example of SERAR recombinant polypeptide successfully perform the transport of polypeptide macromolecules across intestinal epithelial cells.

### Example 4

### Successful of SERAR Amount

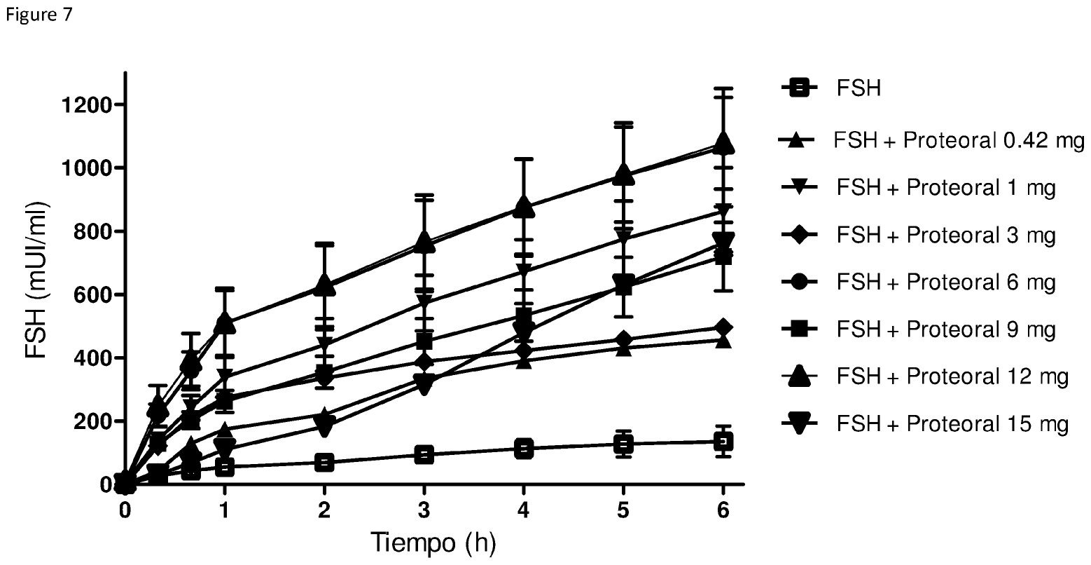

Formulations composed by SERAR and FSH as therapeutic protein, with increasing amounts of SERAR are compared for their ability to facilitate absorption of FSH by transepithelial paracellular passage in methods and compositions of the present invention. SERAR and FSH are co-formulated as described in the above Examples. Increasing amounts of SERAR polypeptide is added to formulations with fixed amount of FSH and those formulations are tested in a Caco-2 monolayer in vitro test. The most effective SERAR polypeptide amount is used in subsequent studies FIG. 7.

### Example 5

### Successful of Protease Inhibitor Amount

Aprotinin protease inhibitor is studied to preserve the SERAR recombinant polypeptide and the therapeutic polypeptide following oral administration in methods and compositions of the present invention. SERAR and the therapeutic polypeptide are co-formulated as described in the above Examples, except that aprotinin is added as a gastroenteric protease inhibitor. The amounts of this protease inhibitor is also varied, to determine the successful amount of gastro-protective protease inhibitor. The most effective protease inhibitor amount is used in Subsequent Examples.

### Example 6

### Successful of Therapeutic Polypeptide FSH Concentration

Formulation are compared for their ability to facilitate absorption of FSH following oral administration in methods and compositions of the present invention. SERAR and FSH are co-formulated as described in the above Examples. The most effective SERAR/FSH is used in Subsequent experiments.

### Example 7

### Manufacture of Polypeptide Nanoparticles Using Purified Recombinant Polypeptide SERAR and Therapeutic Polypeptide Follicle Stimulating Hormone

75 IU of FSH and 25 mg of correctly purified recombinant polypeptide SERAR are dissolved in a final volume of 12.5 mL of polyvinylpyrrolidone (PVP) 0.15% w/v under constant magnetic stirring at 500 rpm. This protein solution is further mixed for 20 min. In the polypeptide nanoparticles synthesis, an appropriate volume of ethanolic solution of gastroprotective methacrylic acid copolymer 0.15% w/v is injected into the protein solution at 3 mL/min using a narrow tube (0.5 mm inner diameter) under constant magnetic stirring at 500 rpm. The SERAR-FSH nanoparticles suspension is kept under magnetic stirring at 500 rpm for another 20 min. Then, one volume of PVP 0.15% w/v is added under magnetic stirring. The nanoparticle suspension, with an average particle diameter between 100-350 nm, is concentrated 5 times followed by a diafiltration using 10 volumes of Tris buffer solution pH 7.4. Both processes are performed by tangential flow filtration using a 300 kDa polyethersulfone membrane (Pellicon 3 Merck-Millipore). The mean particle size remains stable during the concentration and diafiltration. Finally, the protein nanoparticle suspension is lyophilized for 72 h with or without the addition of any lyoprotectant. The mean particle size and polydispersity index remain stable after storing at room temperature and reconstitution with saline solution. The gastro-protected polypeptide nanoparticle synthesis yield (>99%), encapsulation efficiency (>99%) and mean particle size (100-350 nm) are performed as quality control of the reconstituted formulation.

### Example 8

### Extemporaneous Preparation of Gastro-Protected Polypeptide Nanoparticles of Cetuximab

First, 250 mg of cetuximab and 2.5 g of purified recombinant polypeptide SERAR are dissolved in a final volume of 1.4 L of polyvinylpyrrolidone (PVP) 0.15% under constant magnetic stirring at 500 rpm. This protein solution is mixed for 20 min. For the polypeptide nanoparticle synthesis (yield >99%), an appropriate volume of ethanolic solution of Eudragit L-100 0.15% is injected to the protein solution at 3 mL/min using a narrow tube (0.5 mm inner diameter) under stirring at 500 rpm. The SERAR-Cetuximab nanoparticle suspension is kept under magnetic stirring at 500 rpm for 20 min and later. Then, one volume of PVP 0.15% is added under magnetic stirring. The nanoparticle suspension, with an average particle diameter between 200-500 nm, is concentrated 5 times followed by a diafiltration using 10 volumes of Tris buffer solution pH 7.4. Both processes are performed by tangential flow filtration using a 300 kDa polyethersulfone membrane (Pellicon 3 Merck-Millipore). The mean particle size remains stable during the concentration and diafiltration. Finally, the cetuximab nanoparticle dispersion is lyophilized for 72 h with the addition of mannitol as lyoprotectant. The extemporaneous suspension powder composed by SERAR-Cetuximab nanoparticles remains stable—in terms of mean particle size, polydispersity index and encapsulation efficiency—after storing at room temperature and reconstitution with saline solution.

The gastro-protected polypeptide nanoparticle synthesis yield (>99%), encapsulation efficiency (>99%) and mean particle size (100-350 nm) are performed as quality control of the reconstituted formulation.

### Example 9

Microspheres Coated with Gastro-Protected Polypeptide Nanoparticles of Follicle Stimulating Hormone

First, 75 IU (or 150 IU) of Follicle Stimulating Hormone (FSH) and 25.0 mg of purified recombinant polypeptide SERAR are dissolved in a final volume of 10 mL of polyvinylpyrrolidone (PVP) 0.15% under constant stirring at 500 rpm. This polypeptide solution is mixed for 20 min. For the polypeptide nanoparticles synthesis (yield >99%), an appropriate volume of ethanolic solution of Eudragit L-100 0.15% is injected to the protein solution at 2 to 160 mL/min using a narrow nozzle under stirring at 500 rpm. The FSH protein nanoparticle dispersion is kept under stirring at 500 rpm for 20 min and later. One volume of PVP 0.15%, used as stabilizing dilution, is added under magnetic agitation at 500 rpm. The mixture is kept at room temperature for 30 min. The average size of polypeptide nanoparticles are between 250 and 500 nm were obtained. Finally, the polypeptide nanoparticle dispersion is freeze-dried for 48 h with or without the addition of any kind of lyoprotectant (e.g. mannitol).

Gastro-protected FSH polypeptide nanoparticle layering and enteric coating processes in sugar microspheres are carried out with a fluid bed coater Mini Glatt (Glatt®). 50 g of sucrose microspheres (size 710 μm) are weight and put into the Mini Glatt and dried at 35° C. by air current at 40° C. The instrument parameters are:

Coating mode: Würster. Silicon tubes measures: 2 mm (internal diameter) and 4 mm (external diameter).

Sprayer diameter: 0.8 mm.

Inlet air pressure: 0.2-0.4 bar.

Atomization pressure: 1.5-2-5 bar.

Bed temperature: 35-40° C.

Peristaltic pump (Flocon®): 5 rpm.

Protein nanoparticle layering suspension percentage composition

| Ingredient | Commercial name | Percentage (%) |
|---|---|---|
| Therapeutic protein | Freeze dried FSH protein | 0.19 |
| | nanoparticles | |
| PVP K-29/32 | PLASDONE ™ K-29/32 | 6.54 |
| Talc | Van Rossum | 3.27 |
| Distilled water | — | 90 |
| | Total | 100 |

Solid content: 10%.

Once finished the gastro-protected polypeptide nanoparticle layering process the microspheres are dried by air current for 10 min. Then the enteric coating suspension is applied.

### Enteric Coating Percentage Composition

| Ingredient | Commercial name | Percentage (%) |
|---|---|---|
| EUDRAGIT ® L 30 D-55 | EUDRAGIT ® L 30 D-55 | 41.65 |
| Triethyl citrato (TEC) | Citrofol ® AI | 1.25 |
| Talc | Van Rossum | 6.25 |
| Distilled water | — | 50.85 |
| | Total | 100 |

Solid content: 10%.

After the gastro-protective coating, the microspheres are dried again under air current for 10 min. Once the fluid bed coating process is finished, #4 capsules are filled with 130 mg coated microspheres.

1. A universal oral delivery device of intact therapeutic polypeptides to the circulatory system, the device comprising:
a purified recombinant polypeptide, the purified recombinant polypeptide having an amino acid sequence equal to SEQ ID NO. 1 wherein the amino acid in position 560 is a nonpolar amino acid, and
a group consisting of at least one or a combination of two or more therapeutic polypeptide.

2. The universal oral delivery device of claim 1, further comprising a group consisting of at least two gastro-protective polymers.

3. A purified recombinant polypeptide, wherein the purified recombinant polypeptide has an amino acid sequence equal to SEQ ID NO 2.

4. The universal oral delivery device of claim 1, wherein at least one therapeutic polypeptide in the group of therapeutic polypeptides are comprised in the group of: recombinant polypeptides, naturally-existing biologically active proteins, fusion proteins, hormones, growth factors, plasma proteins, coagulation factors, toxins and other protein antigens, monoclonal antibodies, replacement enzymes and peptides.

5. The universal oral delivery device of claim 1, wherein at least one therapeutic polypeptide in the group of therapeutic polypeptides is a monoclonal antibody or a fusion polypeptide including but not limited to rituximab, adalimumab, trastuzumab, ranibizumab, pertuzumab, denosumab, cetuximab, bevacizumab, nivolumab, pembrolizumab, eculizumab, ustekinumab, ab, omalizumab, pembrolizumab, and combinations of two or more of these.

6. The universal oral delivery device of claim 1, wherein at least one therapeutic polypeptide in the group of therapeutic polypeptides is a replacement enzyme comprised in the group of: alfa-agalsidase, alglucerase, beta-agalsidase imiglucerase, taliglucerase-alfa, velaglucerase-alfa, laronidase, idulsurfase, elosulfase-alfa, galsulfase, alglucosidase-alfa.

7. The universal oral delivery device of claim 1, wherein at least one therapeutic polypeptide is a recombinant protein comprised in the group of: follicle stimulating hormone, luteinizing hormone, Chorionic gonadotrophic hormone, erythropoietin, filgrastim, molgramostim, soma pin, betaIFN1a, betaIFN1b, IFNalpha2a, IFNalpha2b, interleukin 2, etanercept, ranibizumab, insulin, eptacog alfa, human recombinant Factor VII, human recombinant Factor VIII, human recombinant Factor XII, human recombinant Factor XIII.

8. The universal oral delivery device of claim 1, wherein at least one polypeptide in the group of therapeutic polypeptides is included in therapeutic amounts.

9. The oral delivery device of claim 1, wherein the purified recombinant polypeptide and the group of therapeutic polypeptides are formulated as gastro-protected polypeptide nanoparticles.

10. An isolated nucleic acid comprising a sequence encoding a recombinant polypeptide according to claim 3 or a fragment of that nucleic sequence of at least 18 consecutive bases.

11. An expression vector for an isolated nucleic acid comprising
the isolated nucleic acid according to claim 10, wherein the recombinant sequence is operably linked to an expression control sequence.

12. A cultured cell comprising an expression vector as claimed in claim 11.

13. A cultured cell comprising an isolated nucleic acid according to claim 10, wherein the recombinant sequence is operably linked to an expression control sequence.

14. A cultured cell or a progeny of said cell,
wherein the cultured cell is transfected with an expression vector for an isolated nucleic acid, the isolated nucleic acid comprising the isolated nucleic acid according to claim 10, and
wherein the recombinant sequence is operably linked to an expression control sequence, and the cultured cell expresses the recombinant polypeptide.

15. (canceled)

16. (canceled)

17. A method for synthesizing gastro-protected polypeptide nanoparticles suspension, the gastro-protected polypeptide nanoparticles formulated from a purified recombinant polypeptide having an amino acid sequence equal to SEQ ID NO. 1,
wherein the amino acid in position 560 is a nonpolar amino acid, and a group consisting of at least one or a combination of more therapeutic polypeptides, the method comprising solvent-injecting a solution containing a gastro-protective polymer in ethanol into an aqueous solution, the aqueous solution containing the gastro-protected polypeptide nanoparticles, one or more therapeutic polypeptides, and a stabilizing ent.

18. The method of claim 17, wherein the gastro-protected polypeptide nanoparticles are shell-coated.

19. The method of claim 17, wherein the average diameter size of the polypeptide nanoparticles generated is between approximately 250 and 350 nanometers.

20. The method of claim 17, wherein the solvent-injecting of the ethanolic solution into the aqueous solution is done using a narrow tube made of steel and having an internal diameter between approximately 0.5 and 20 millimeters.

21. (canceled)

22. The method of claim 17, wherein the ethanolic solution is injected at a flow rate between approximately 0.5 and 5000 milliliters per minute, or between approximately 2 and 160 millimeters.

23. The method of claim 17, further comprising the step of diluting the gastro-protected polypeptide nanoparticles with the addition of a stabilizing agent.

24. (canceled)

25. (canceled)

26. The method of claim 17, wherein the polypeptide s suspension is freeze-dried.

27. The method of claim 17, wherein the polypeptide nanoparticles suspension is diafiltered and concentrated up to 10 times.

28. (canceled)

29. The method of claim 27, further comprising the step of adding a lyoprotectant agent to the polypeptide nanoparticles suspension before freeze drying.

30. (canceled)

31. (canceled)

32. (canceled)

33. The method of claim 17, further comprising the step of removing any water miscible organic solvent from the synthesized gastro-protected polypeptide nanoparticles,
wherein the removal is done by one of dialysis, ultra-filtration, solvent evaporation at a reduced pressure, N₂ current evaporation or tangential flow filtration.

34. A pharmaceutical composition of gastro-protected polypeptide nanoparticles, the gastro-protected polypeptide nanoparticles formulated from a purified recombinant polypeptide having an amino acid sequence equal to SEQ ID NO. 1, wherein the amino acid in position 560 is a nonpolar amino acid, and a group consisting of at least one or a combination of more therapeutic polypeptides in oral dosage forms as syrups, solutions, ampoules, dispersions, semi-solids, softgels, tablets, capsules, sachets, powders, granules, orally dispersible films.

35. (canceled)

36. A method of oral administration of a pharmaceutical composition to a subject, the method comprising:
administering orally to said subject the pharmaceutical composition according to claim 34.
